(19) United States
(12) Patent Application Publication
(10) Pub. No.: US 2014/0308242 A1
(43) Pub. Date: Oct. 16, 2014

(54) BIOMARKERS FOR HCV INFECTED PATIENTS

(71) Applicants: Mark Andrew Fleming, Winchester, MA (US); Brain J. Hare, Arlington, MA (US); Daniel Chelsky, Westmount (CA); Rene Allard, Montreal (CA); Eustache Paramithiotis, Boucherville (CA)

(72) Inventors: Mark Andrew Fleming, Winchester, MA (US); Brain J. Hare, Arlington, MA (US); Daniel Chelsky, Westmount (CA); Rene Allard, Montreal (CA); Eustache Paramithiotis, Boucherville (CA)

(73) Assignee: VERTEX PHARMACEUTICALS INCORPORATED, Cambridge, MA (US)

(21) Appl. No.: 13/879,736

(22) PCT Filed: Oct. 21, 2011
(86) PCT No.: PCT/US2011/057347

§ 371 Date: Sep. 3, 2013

Related U.S. Application Data
(60) Provisional application No. 61/405,619, filed on Oct. 21, 2010.

(51) Int. Cl. G01N 33/53; A61K 38/08; A61K 31/7056; G01N 33/573; C12Q 1/48; C12Q 1/32; A61K 38/21

(52) U.S. Cl. CPC G01N 33/53; C12Q 1/32; A61K 38/08; A61K 38/21; G01N 33/573; C12Q 1/485; C12Q 1/48; A61K 31/7056; G06F 19/3431
USPC 424/85.4; 435/26; 435/7.92; 514/4.3; 435/7.4; 435/23; 435/15; 702/19

(57) ABSTRACT

The invention relates to biomarkers measurable in a human subject that have prognostic value with respect to efficacy of therapeutic treatments for Hepatitis C viral infection. The markers also are believed to have value for diagnosis liver health/liver damage.

### CROSS-REFERENCE TO RELATED APPLICATIONS

This application claims priority to U.S. Provisional Patent Application No. 61/405,619, filed Oct. 21, 2010, the disclosure of which is incorporated by reference in its entirety.

### TECHNICAL FIELD OF THE INVENTION

The invention relates to biomarkers having prognostic value with respect to efficacy of therapeutic treatments for Hepatitis C viral infection and diagnosis of liver health/liver damage.

### INCORPORATION BE REFERENCE OF MATERIAL SUBMITTED ELECTRONICALLY

Incorporated by reference in its entirety is a computer-readable nucleotide/amino acid sequence listing submitted concurrently herewith and identified as follows: ASCII text file named “45796A_PCT_SeqListing.txt”, 8,490 bytes, created 21 Oct. 2011.

### BACKGROUND

Hepatitis C is an infectious disease affecting the liver, caused by the hepatitis C virus (HCV). The disease affects more than 4 million people in the United States and hundreds of millions of people worldwide. Infection by hepatitis C virus (“HCV”) is recognized as the causative agent for most cases of non-A, non-B hepatitis, with an estimated human sero-prevalence of 3% globally (Alberti et al., J. Hepatology, 31 (Suppl. 1), 17-24 (1999)).

Upon first exposure to HCV, only about 20% of infected individuals develop acute clinical hepatitis; others appear not to develop significant outward symptoms of infection. The infection often is asymptomatic, perhaps for many years or decades, but once established, chronic infection can progress to scarring of the liver (fibrosis), advanced scarring (cirrhosis); liver failure; liver cancer; and death. The virus persists in about 85% of infected individuals, and can be spread by blood-to-blood contact. Acute HCV infection refers to the first six months of infection with the HCV virus, whereas chronic HCV infection refers to infection persisting more than six months. It is not uncommon for HCV to go undiagnosed during the acute phase, and to be asymptomatic, at least initially, in the chronic phase.

HCV comprises a single-stranded positive-sense RNA genome encoding a polyprotein of 3010-3033 amino acids, which is co- or post-translationally processed into structural proteins (e.g., core, E1, and E2) and nonstructural (NS) proteins (e.g., NS2, NS3, NS4A, NS4B, NS5A, and NS5B) (Choo et al., Proc. Natl. Acad. Sci. USA, 88, 2451-2455 (1991); Kato et al., Proc. Natl. Acad. Sci. USA, 87, 9524-9528 (1990); Takamizawa et al., J. Virol., 65, 1105-1113 (1991); Choo et al., Science, 244, 359-362 (1989)). Host peptidase first cleaves the polyprotein to release the structural proteins (Hijikata et al., Proc. Natl. Acad. Sci. USA, 88, 5547-5551 (1991); Lin et al., J. Virol., 68, 5063-5073 (1994)). The NS2/3 metalloprotease cleaves at the NS2/NS3 junction. NS3 (with cofactor NS4A) displays serine protease activity and further processes the viral polyprotein to generate the majority of the viral enzymes essential for viral replication and infectivity, including NS4B, NS5A, and NS5B proteins (Bartenschlager et al., J. Virol., 67, 3835-3844 (1993)).

All nonstructural proteins play a role in HCV replication and/or packaging, and antiviral agents targeting of NS3 protease and NS5B polymerase have shown a great deal of promise in the clinic. NS5B is an RNA-dependent RNA polymerase (RdRp) and terminal transferase, and plays a key role in replication of the viral RNA genome (Lohmann et al., J. Virol., 71, 8416-8428 (1997); Lohmann et al., Virology, 249, 108-118 (1998); Kolykhalov et al., J. Virol., 74(4), 2046-2051 (2000)). The NS5B protein comprises approximately 591 amino acids (65 kDa) having canonical motifs common to other RNA viral polymerases.

No effective HCV vaccine exists to date, and pegylated interferon alpha (IFN-alpha-2a or IFN-alpha-2b) and ribavirin (brand names: Copegus, Rebetol, Ribasphere, Vilona and Virazole) combination therapy is currently the standard of care (SoC) for the treatment of chronic hepatitis C, although numerous efforts exist for improved therapies. With this standard of care therapy, sustained virologic responses (SVR)—defined as undetectable HCV RNA in the serum—has been achieved in greater than 50% of chronic HCV subjects. However, the course of treatment is long (24 or 48 weeks), and the SoC therapy is associated with a high incidence of side effects, including flu-like symptoms and hematologic complications such as neutropenia and thrombocytopenia due to the interferon and hemolysis/anemia due to the ribavirin. The long course of therapy and adverse side effects impact patient compliance with therapy and the effectiveness of the therapy. Some doctors and patients choose to delay SoC therapy until an infected subject begins to show signs of liver damage.

The National Institutes of Health Consensus Development Conference Panel recommended that therapy for hepatitis C be limited to those patients who have histological evidence of progressive disease. Thus, the panel recommended that all patients with fibrosis or moderate to severe degrees of inflammation and necrosis on liver biopsy should be treated and that patients with less severe histological disease be managed on an individual basis. Patient selection should not be based on the presence or absence of symptoms, the mode of acquisition, the genotype of HCV RNA, or serum HCV RNA levels. Proceedings of the June 10-12 “Management of Hepatitis C: 2002. National Institutes of Health Consensus Development Conference Update.” Hepatology. 2002; 36(5, part 2). Improved metrics to evaluate the merits of these recommendations would benefit HCV therapy decisions.

In view of the potential severe complications of HCV infection, the high cost of therapy in terms of both financial outlay and quality of life, and the failure of SoC therapy to achieve a cure in close to 50% of chronically infected HCV patients, a need exists to identify improved parameters for making treatment decisions for HCV-infected patients.

### SUMMARY OF THE INVENTION

The present invention provides materials, methods, and systems that will improve healthcare related to diseases related to the liver, and in particular improve HCV management and therapy.

For example, some aspects of the invention relate to methods of evaluating a human subject infected with hepatitis C virus (HCV) for a treatment. Such methods are useful, for example, for selecting which HCV patients will benefit from which HCV therapies, and/or when they will be most likely to benefit from the therapy, and/or selecting which therapy is most likely to benefit a patient, if more than one therapy is available.

In one variation, the invention includes a method of evaluating a human subject infected with hepatitis C virus (HCV) for a treatment, the method comprising: (a) obtaining measurement(s) of at least one biomarker from at least one biological sample isolated from a human subject who is infected with HCV; and (b) evaluating the subject for HCV treatment from the output of a model, wherein the inputs to said model comprise said measurement(s) of the at least one biomarker. All of the biomarkers described herein are candidate markers useful for practicing the invention. In some variations, the at least one biomarker is selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof. As set forth in Example 1 below, each of these markers showed differential expression in subjects that achieved a sustained viral response benefit from a therapy that comprised Telaprivir, compared to subjects that did not achieve this level of response. In other variations, the at least one biomarker is selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof. As set forth below in Example 1, each of these markers showed differential expression in subjects that achieved a sustained viral response benefit from a current standard of care therapy that comprised an interferon and Ribavirin (but without Telaprivir), compared to subjects that did not achieve this level of response.

As set forth in the Examples, some of the differentially expressed markers are more highly expressed in HCV-infected subjects that achieved sustained viral response to the therapy, whereas other markers were more less highly expressed in these patients, compared to HCV-infected subjects that did not achieve the same beneficial response to the therapy. In variations of the invention in which a single marker is used in the model, then a cutoff or reference value may be sufficient to serve as a comparison or reference point. For multimarker analysis, statistical analysis of express data can be used for model building and patient analysis.

In some variations of the invention, the model is developed by fitting data from a study of a population of human subjects who were infected with HCV and who received at least treatment for the HCV. For example, in some variations, the treatment for the human subjects in the study comprises administration of an interferon, alone or in combination with another agent. In some variations, the interferon is a pegylated interferon alpha. In some variations, the treatment for the human subjects in the study comprises administration of ribavirin.

In still other variations of the invention, the treatment for the human subjects in the study comprises administration of telaprevir. The telaprevir may be administered as a single therapeutic agent or in combination with other agents, such as an interferon or ribavirin.

In some variation of the method, the at least one biomarker includes a biomarker for which a change in biomarker serum concentration correlates with the presence or severity of liver damage. The experiments described in Example 1 identify many such markers. For example, in some variations, the at least one biomarker is selected from the group consisting of A2M, CD5L, LGALS3BP, CNDP1, and CLEC3B. In some preferred variations, the at least one biomarker comprises CNDP1, or comprises LGALS3BP, or comprises both of these markers.

In some variations of the invention, additional factors besides the biomarkers are included in the analysis of the patient and in the modeling to evaluate the likelihood of success of a particular therapy. For example, in some variations, the method further comprises determining an HCV genotype of the HCV that infects the human subject, wherein the inputs to the model further comprise the HCV genotype, and wherein the study of the population of human subjects included data collection about HCV genotype.

In some variations, the method further comprises obtaining measurements from the at least one biological sample of at least one supplemental biomarker selected from the group consisting of apolipoprotein A1, haptoglobin, total bilirubin, and γ-glutamyl-transpeptidase (GGT), wherein the inputs of the model further include the measurement(s) of the at least one supplemental biomarker, and wherein the study of the population of human subjects included data collection about the at least one supplemental biomarker. These supplemental markers are included in the FIBROTEST model.

In some variation, the method further comprises obtaining a measurement of at least one clinical parameter of the subject that is relevant to HCV treatment. Exemplary clinical parameters that are specifically contemplated include: sex (gender) of the subject, age, race or ethnicity (which can be self-reported or genetically analyzed), weight, body mass index, height, weight, hip circumference, waist circumference, history (past and current) of tobacco usage, history (past and current) of alcohol consumption, exercise pattern, presence of diabetes, fasting glucose, triglycerides, fibrosis score, and HCV viral load, for example. The inputs of the model further include the measurement(s) of the at least one clinical parameter, and the study of the population of human subjects includes data collection about the at least one clinical parameter.

Clinical analysis has shown that the success rate of Telaprivir and other HCV therapies can be different, e.g., lower, in African Americans than in Caucasians. In some variations of the invention, the race is taken into account in the modeling. For example, in some variations, the subject is Caucasian. In some variations, the model is also based on inputting of race as a stratifying factor for the data.

In still further variations, the method further comprises obtaining a measurement of alanine transaminase (ALT), Aspartate Aminotransferase (AST), and combinations thereof from the at least one biological sample, wherein the inputs of the model further include the measurement of ALT and/or AST, and wherein the study of the population of human subjects included data collection about ALT and/or AST. These are traditional markers evaluated in connection with liver health.

In still further variations, the method further comprises obtaining a measurement of Carbohydrate-deficient transferrin (CDT) from the at least one biological sample, wherein the inputs of the model further include the measurement of CDT, and wherein the study of the population of human subjects included data collection about CDT. CDT can provide clinical information about past alcohol use.

In some variations, the method further comprises determining liver stiffness, wherein the inputs of the model further include measurement of liver stiffness of the subject, and wherein the study of the population of human subjects included data collection about liver stiffness. Liver stiffness is the focus of, e.g., the FIBROSCAN test.

Some variations of the invention include further steps in addition to the obtaining/evaluating steps described above. For example, in some variations, the method of the invention further comprises a step, prior to the measuring the biomarkers, of obtaining at least one biological sample from the subject. Exemplary samples include whole blood or blood components such as serum or plasma. The biological sample could be other tissues or fluids, including tissue obtain by biopsy, such as liver biopsy.

In some variations of the method of the invention, the obtaining measurements comprises measuring at least one of the biomarkers in the at least one biological sample. The at least one of said biomarker measurements can be obtained by an immunoassay, for example. If at least one of said biomarkers is an enzyme, then the biomarker optionally is measured by an enzymatic activity assay.

In still further variations of the invention, the obtaining of biomarker measurement data comprises obtaining data representative of a measurement of the at least one biomarker from a preexisting record. Optionally, some biomarker measurement data is obtained from a record and other marker measurement data is obtained by measuring in the biological sample.

The method optionally includes one or more additional steps that occur post-evaluation. For example, in some variations, the method further includes (c) reporting said evaluation to a reporting machine comprising a visual display (such as a computer monitor or video display screen), a speaker, or a printer. In some variations, the method further includes a step of storing the evaluation on a paper or an electronic data storage medium. In some variations, the method further comprises a step of advising said human subject, or a health care practitioner, of said evaluation.

It will be appreciated that the biomarkers described herein are useful for predicting what patients are more likely to benefit for an HCV therapy. Conversely, the methods also are useful for predicting which patients are less likely to benefit from the same therapy. Thus, in some variations of the invention, the evaluation comprises a determination of an elevated probability of achieving sustained viral response (SVR) from the treatment for HCV infection. In this context, the term “elevated” is relative to the percentage of HCV-infected patients as a whole (without regard to biomarker stratification) that achieve SVR in response to the same therapy. In some variations, the evaluation or the elevated probability can be expressed as a numerical likelihood that the treatment will be successful. For example, in some variations of the method, the elevated probability may be at least 65%, at least 70%, at least 75%, at least 80%, at least 85%, at least 87%, at least 89%, at least 90%, at least 91%, at least 92%, at least 93%, at least 94%, or at least 95% probability of sustained viral response (SVR) six months after cessation of the therapy. Alternatively, the likelihood of successful therapy can be expressed by way of some other indexing, such as stratifying subjects into quartiles, quintiles, letter grades, or other divisions based on likelihood that a treatment will achieve at least SVR.

In still further variations, the invention includes methods that include administering an HCV treatment to a subject that is identified, according to an evaluation tool described herein, as likely to benefit from the HCV treatment. For example, in one variation, the method of evaluating the subject is performed, and the method further includes a step of administering to the subject an HCV treatment. Preferably, the treatment is a treatment identified as having an elevated likelihood of success at achieving sustained viral response (or cure) for that subject, based on the evaluation of biomarker(s).

A related aspect of the invention is a method of treating HCV infection in a human subject comprising: (a) measuring at least one marker in a biological sample from a human subject infected with HCV, wherein the at least one marker is selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof; and (b) administering a composition comprising telaprevir to the subject if the measurement(s) of the at least one biomarker indicates a probability of at least 65%, at least 70%, at least 75%, at least 80%, at least 85%, at least 87%, at least 89%, at least 90%, at least 91%, at least 92%, at least 93%, at least 94%, or at least 95% of sustained viral response (SVR) six months after cessation of the therapy.

A further aspect of the invention is a method of treating HCV infection in a human subject comprising: (a) measuring at least one marker in a biological sample from a human subject infected with HCV, wherein the at least one marker is selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof; and (b) administering a treatment comprising an interferon and ribavirin to the subject if the measurement(s) of the at least one biomarker indicates a probability of at least 65%, at least 70%, at least 75%, at least 80%, at least 85%, at least 87%, at least 89%, at least 90%, at least 91%, at least 92%, at least 93%, at least 94%, or at least 95% of sustained viral response (SVR) six months after cessation of the therapy.

In a related aspect, the invention is a method of treating hepatitis C infection in a human subject, the method comprising:
obtaining an evaluation representing a likelihood that a human subject infected with HCV will benefit from an HCV therapeutic regimen, wherein the evaluation is computed according to any of the methods described herein; and
generating prescription treatment data for the subject representing a prescription for a treatment regimen for HCV with a likelihood of sustained viral response (SVR) six months after cessation of the therapy for the subject of at least 65%, at least 70%, at least 75%, at least 80%, at least 85%, at least 87%, at least 89%, at least 90%, at least 91%, at least 92%, at least 93%, at least 94%, or at least 95% probability.
   In some variations, such a method further comprises administering the therapeutic regimen to the subject.

Another related aspect of the invention is a method of therapy for HCV infection, the method comprising:
evaluating the likelihood that a human subject infected with HCV will benefit from an HCV therapeutic regimen according to the method of any one of claims 1-29; and
treating the subject according to the therapeutic regimen if the likelihood of sustained viral response (SVR) six months after cessation of the therapy for the subject of at least 65%, at least 70%, at least 75%, at least 80%, at least 85%, at least 87%, at least 89%, at least 90%, at least 91%, at least 92%, at least 93%, at least 94%, or at least 95% probability.

The invention further includes materials that are useful for practicing methods of the invention. For instance, the invention includes kits that contain materials useful for performing the evaluations described herein, packaged together to assist a clinician or laboratory.

In one variation, the invention is a kit comprising reagents for measuring at least 2, at least 3, at least 4, at least 5, at least 6, at least 7, at least 8, at least 9, or at least 10 biomarkers packaged together, wherein the biomarkers are selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof.

In another variation, the invention is a kit comprising reagents for measuring at least 2, at least 3, at least 4, at least 5, at least 6, at least 7, at least 8, at least 9, or at least 10 biomarkers packaged together, wherein the biomarkers are selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof.

Exemplary reagents for measuring protein biomarkers include primary antibodies that bind to the biomarker, optionally labeled or optionally attached to a solid support. For measuring enzyme biomarkers, the reagent may include a substrate for an enzymatic activity assay. Optionally, the kit further includes a buffer and/or instructions for performing the assay. In some variations, the kit further comprising molecular standards for the biomarkers.

Still another variation of the invention is a computer readable medium having computer executable instructions for evaluating an HCV-infected subject for a treatment, the computer readable medium comprising:
a routine, stored on the computer readable medium and adapted to be executed by a processor, to store biomarker and clinical measurement data representing measurements of at least one biomarker; and a routine stored on the computer readable medium and adapted to be executed by a processor to analyze the biomarker measurement data to evaluate likelihood of success of a therapy for hepatitis C for a human subject infected with hepatitis C virus (HCV),
wherein the at least one biomarker is selected from the group consisting of LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof.

A related variation of the invention is a computer readable medium having computer executable instructions for evaluating an HCV-infected subject for a treatment for the HCV infection, the computer readable medium comprising:
a routine, stored on the computer readable medium and adapted to be executed by a processor, to store biomarker and clinical measurement data representing measurements of at least one biomarker; and a routine stored on the computer readable medium and adapted to be executed by a processor to analyze the biomarker measurement data to evaluate likelihood of success of a therapy for hepatitis C for a human subject infected with hepatitis C virus (HCV),
wherein the at least one biomarker is selected from the group consisting of carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof.

In some variations of the computer readable media of the invention, the biomarker and clinical measurement data includes data representing measurements of at least one clinical parameter selected from the group consisting of: sex, age, race, weight, body mass index, height, weight, hip circumference, waist circumference, history of tobacco usage, history of alcohol consumption, exercise pattern, presence of diabetes, fasting glucose, triglycerides, fibrosis score, and HCV viral load. In other variations, the computer readable media include data representing any of the other types of measurements described herein, e.g., with respect to methods of the invention.

Another aspect of the invention is diagnostic test systems. For example, the invention includes a medical diagnostic test system for evaluating likelihood of benefit of a therapy for hepatitis C in a human subject infected with hepatitis C virus (HCV), the system comprising:
a data collection tool adapted to collect biomarker and clinical measurement data representative of measurements of biomarkers and clinical parameters from a human subject, wherein said biomarkers comprise at least one marker selected from the group consisting of: wherein the at least one biomarker is selected from the group consisting of LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof;
an analysis tool comprising a statistical analysis engine adapted to generate a representation of a correlation between likelihood of benefit from the therapy and measurements of the biomarkers and clinical parameters, wherein the representation of the correlation is adapted to be executed to generate a result; and
an index computation tool adapted to analyze the result to determine the human subject's likelihood of benefitting from the therapy and represent the result as a numerical probability or a grade or score.

A related aspect of the invention is a medical diagnostic test system for evaluating likelihood of benefit of a therapy for hepatitis C in a human subject infected with hepatitis C virus (HCV), the system comprising:
a data collection tool adapted to collect biomarker and clinical measurement data representative of measurements of biomarkers and clinical parameters from a human subject, wherein said biomarkers comprise at least one marker selected from the group consisting of: wherein the at least one biomarker is selected from the group consisting of carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof;
an analysis tool comprising a statistical analysis engine adapted to generate a representation of a correlation between likelihood of benefit from the therapy and measurements of the biomarkers and clinical parameters, wherein the representation of the correlation is adapted to be executed to generate a result; and
an index computation tool adapted to analyze the result to determine the human subject's likelihood of benefitting from the therapy and represent the result as a numerical probability or a grade or score.

The diagnostic test system optionally includes a data collection tool, analysis tool, and index computation tool that is further adopted to collect and process the other types of patient information described herein with respect to methods of the invention (e.g., clinical parameters, supplemental markers, etc.).

In some variations of the invention, the system further comprises a reporting tool adapted to generate a report comprising the numerical probability, grade, or score.

The invention further includes methods of developing models for evaluating the likelihood that a hepatitis-infected patient will benefit from a treatment, using biomarker inputs and optionally any of the other types of inputs described herein with respect to methods of the invention.

For example, the invention includes a method of developing a model for evaluation likelihood that a human subject infected with hepatitis C virus (HCV) will benefit from an HCV therapeutic regimen, the method comprising:
obtaining biomarker and clinical measurement data, wherein the biomarker and clinical measurement data is representative of measurements of biomarkers and clinical parameters from a population of humans infected with HCV, and includes endpoints of the population; wherein said biomarkers and clinical parameters for which measurement data is obtained comprise at least one marker selected from the group consisting of LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof;
inputting the biomarker and clinical measurement data of at least a subset of the population into a model; and
training the model for endpoints using the inputted biomarker and clinical measurement data to derive a representation of a correlation between a likelihood of benefit from the therapeutic regimen and measurements of biomarkers and clinical parameters from a human subject.

Similarly, the invention includes a method of developing a model for evaluation likelihood that a human subject infected with hepatitis C virus (HCV) will benefit from an HCV therapeutic regimen, the method comprising:
obtaining biomarker and clinical measurement data, wherein the biomarker and clinical measurement data is representative of measurements of biomarkers and clinical parameters from a population of humans infected with HCV, and includes endpoints of the population; wherein said biomarkers and clinical parameters for which measurement data is obtained comprise at least one marker selected from the group consisting of carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof;
inputting the biomarker and clinical measurement data of at least a subset of the population into a model; and
training the model for endpoints using the inputted biomarker and clinical measurement data to derive a representation of a correlation between a likelihood of benefit from the therapeutic regimen and measurements of biomarkers and clinical parameters from a human subject.

Some aspects of the invention can be characterized as new uses of materials. For example, an aspect of the invention is the use of at least one antibody or other specific binding construct for evaluating whether a human subject who is infected with HCV will benefit from a treatment for HCV infection, wherein the at least one antibody binds to a marker selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; and fragments of any of the foregoing biomarkers that contain an epitope or peptide sequence specific for the biomarker. Still another aspect of the invention is the use of at least one antibody or other specific binding construct for evaluating whether a human subject who is infected with HCV will benefit from a treatment for HCV infection, wherein the at least one antibody binds to a marker selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); and fragments of any of the foregoing biomarkers that contain an epitope or peptide sequence specific for the biomarker.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. For example, where embodiments concerning a method of diagnosis or evaluation are described, embodiments involving methods of therapy, kits, computer readable media, diagnostic systems, and the like that have the same properties and features are specifically contemplated, and the reverse also is true. Where embodiments of the invention are described with respect to a specific biomarker, it should be appreciated that analogous embodiments involving fragments of the biomarker, or nucleic acids (e.g., mRNA) that encode the biomarker and whose concentration in a biological sample may vary in predictable ways with the concentration of the marker itself.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to elements described as a selection of one or more (or at least one) within a set, it should be understood that all combinations within the set are contemplated. For instance, if a method involves evaluation of at least one biomarker from a set of biomarkers, it should be understood that each combination of biomarkers in the set is specifically contemplated as a different embodiment of the invention.

With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a patent office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE DRAWINGS

FIG. 1 depicts the intensities for HCV-infected subjects of the four peptides identified in Example 1 that were the best predictors of response to the standard of care (SOC) HCV treatment. The subjects are ordered with nonresponders on the left and responders on the right. The Y-axis is scaled related to the intensity of the peptides detected by the mass spectrometer.

FIG. 2 illustrates an example of a suitable computing system environment 100 on which a system for the steps of the claimed method and apparatus may be implemented.

FIG. 3 is a flow diagram of an example method for developing a model which may be used to evaluate a person, or group of people, for likelihood of response to an HCV therapeutic regimen.

FIG. 4 is a flow diagram of an example method for using a model to evaluate an HCV-infected subject (e.g., a person, or group of people) for response to an HCV therapeutic regimen.

### DETAILED DESCRIPTION

The invention is described in further detail below. Section headings are for convenience of reading and not intended to be limiting per se.

Proteins Biomarkers

All proteins that are differentially expressed in human HCV-infected subjects that are differentially expressed in subjects who respond to a therapeutic regimen, compared to subjects who do not, are contemplated as biomarkers for use in the invention. Many such biomarkers are identified throughout the application, including the Examples. It will be appreciated that if a protein represents a useful biomarker, then there may be equivalent biomarkers that are also contemplated for use in the invention, including fragments/metabolites of the same protein, and mRNA that encode the protein.

As described in greater detail in the Examples, experiments designed to identify biomarkers to distinguish HCV patients who achieve a sustained viral response from those who do not identified a total of 34 proteins that distinguish Caucasian Telaprevir-treated patients who achieved SVR from those who did not:

| Gene Symbol | Gene ID |
|---|---|
| LPA | 4018 |
| CNDP1 | 84735 |
| TPM4 | 7171 |
| GAPDH | 2597 |
| FKBP1A | 2280 |
| PARVB | 29780 |
| VCP | 7415 |
| PPIA | 5478 |
| PFN1 | 5216 |
| CAP1 | 10487 |
| ILK | 3611 |
| PLEK | 5341 |
| GSTP1 | 2950 |
| TLN1 | 7094 |
| ZYX | 7791 |
| CLIC1 | 1192 |
| F13A1 | 2162 |
| VCL | 7414 |
| FLNA | 2316 |
| SDPR | 8436 |
| TAGLN2 | 8407 |
| C9 | 735 |
| CP | 1356 |
| YWHAE | 7531 |
| ORM1 | 5004 |
| HPR | 3250 |
| FERMT3 | 83706 |
| A2M | 2 |
| SERPINA1 | 5265 |
| LGALS3BP | 3959 |
| CTSD | 1509 |
| FTL | 2512 |
| CHI3L1 | 1116 |
| FCGBP | 8857 |

These markers, alone or in all combinations of two or more, are contemplated as biomarkers useful for methods, kits, and the like of the invention.

The same sets of experiments also identified a partially overlapping set of ten biomarkers that are useful for identifying and distinguishing those HCV patients that achieve sustained viral response from a standard of care therapy (pegylated interferon alpha and ribavirin) from those that do not achieve this level of benefit from the same therapy:

| Gene Symbol | Gene ID |
|---|---|
| CNDP1 | 84735 |
| TLN1 | 7094 |
| PZP | 5858 |
| APOC4 | 346 |
| CLEC3B | 7123 |
| APOB | 338 |
| A2M | 2 |
| LGALS3BP | 3959 |
| FCGBP | 8857 |
| CD5L | 922 |

These markers, alone or in all combinations of two or more, are contemplated as biomarkers useful for methods, kits, and the like of the invention.

Carnosine dipeptidase 1 is a member of the metallopeptidase M20 family (Gene ID: 84735; Official Full Name carnosine dipeptidase 1 (metallopeptidase M20 family); Primary source HGNC:20675; Locus tag UNQ1915/PRO4380; also known as CN1; CPGL2; HsT2308; MGC10825; MGC102737; MGC142072; CNDP1; NCBI Reference Sequence: NM_{—}032649.5; Swiss-Prot: Q96KN2). The metalloprotease protein encoded by this gene is specifically expressed in the brain, and is a homodimeric dipeptidase which was identified as human carnosinase. Studies of CNDP1 have implicated the protein in nephropathy, hyperglycemia and diabetes. Antibodies to this protein are sold by Sigma-Aldrich and R&D Systems.

Talin 1 (TLN1) (Gene ID: 7094; Primary source HGNC:11845; Locus tag RP11-112J3.1; also known as TLN; ILWEQ; KIAA1027; TLN1; NCBI Reference Sequence: NM_{—}006289.3) encodes a cytoskeletal protein that is concentrated in areas of cell-substratum and cell-cell contacts. The encoded protein plays a significant role in the assembly of actin filaments and in spreading and migration of various cell types, including fibroblasts and osteoclasts. It co-distributes with integrins in the cell surface membrane in order to assist in the attachment of adherent cells to extracellular matrices and of lymphocytes to other cells. The N-terminus of TLN1 protein contains elements for localization to cell-extracellular matrix junctions. The C-terminus contains binding sites for proteins such as beta-1-integrin, actin, and vinculin.

Pregnancy-zone protein (PZP) (Primary source HGNC:9750; also known as CPAMD6; MGC133093; PZP; NCBI Reference Sequence: NM_{—}002864.2) is a major pregnancy-associated plasma protein having a structure similar to that of human alpha 2-macroglobulin and known to interact with proteinases and methylamine.

Apolipoprotein C-IV (APOC4; Gene ID: 346; Primary source HGNC:611; NCBI Reference Sequence: NM_{—}001646.1) is a member of the apolipoprotein gene family. It is expressed in the liver and has a predicted protein structure characteristic of other genes in this family. Apo C4 is a 3.3-kb gene consisting of 3 exons and 2 introns; it is located 0.5 kb 5′ to the APOC2 gene.

C-type lectin domain family 3, member B (CLEC3B; Gene ID: 7123; Primary source HGNC:11891; Swiss Prot Accession No: P05452; also known as Tetranectin (TN); TNA; DKFZp686H17246; and Plasminogen kringle 4-binding protein; NCBI Reference Sequence: NM_{—}003278.2) is a gene that encodes a protein that binds to plasminogen and to isolated kringle 4. It may be involved in the packaging of molecules destined for exocytosis.

Apolipoprotein B (including Ag(x) antigen (APOB; Gene ID: 338; Primary source HGNC:603; Swiss-Prot PO₄₁₁₄; NCBI Reference Sequence: NM_{—}000384.2; also known as FLDB; LDLCQ4; Apolipoprotein B-100) encodes a gene product that is the main apolipoprotein of chylomicrons and low density lipoproteins. It occurs in plasma as two main isoforms, apoB-48 and apoB-100: the former is synthesized exclusively in the gut and the latter in the liver. The intestinal and the hepatic forms of apoB are encoded by a single gene from a single, very long mRNA. The two isoforms share a common N-terminal sequence. The shorter apoB-48 protein is produced after RNA editing of the apoB-100 transcript at residue 2180 (CAA->UAA), resulting in the creation of a stop codon, and early translation termination. Mutations in this gene or its regulatory region cause hypobetalipoproteinemia, normotriglyceridemic hypobetalipoproteinemia, and hypercholesterolemia due to ligand-defective apoB, diseases affecting plasma cholesterol and apoB levels.

Alpha-2-macroglobulin (A2M; Gene ID: 2; Primary source HGNC:7; NCBI Reference Sequence: NM_{—}000014.4; Swiss-Prot P01023; also known as CPAMD5; FWP007; S863-7; DKFZp779B086) is a protease inhibitor and cytokine transporter. It inhibits many proteases, including trypsin, thrombin and collagenase. A2M is implicated in Alzheimer disease (AD) due to its ability to mediate the clearance and degradation of Amyloid-beta, the major component of beta-amyloid deposits.

Lectin, galactoside-binding, soluble, 3 binding protein (LGALS3BP; Gene ID: 3959; Swiss-Prot Q08380 (LG3BP_HUMAN); NCBI Reference Sequence: NM_{—}005567.3; also known as Basement membrane autoantigen p105; BTBD17B; MAC2BP; MAC-2-BP; Mac-2-binding protein; L3 antigen; Tumor-associated antigen 90K; galectin-3-binding protein) is a member of the galectin family of proteins. The galectins are a family of beta-galactoside-binding proteins implicated in modulating cell-cell and cell-matrix interactions. LGALS3BP has been found elevated in the serum of patients with cancer and in those infected by the human immunodeficiency virus (HIV). It appears to be implicated in immune response associated with natural killer (NK) and lymphokine-activated killer (LAK) cell cytotoxicity. Using fluorescence in situ hybridization the full length 90K cDNA has been localized to chromosome 17q25. The native protein binds specifically to a human macrophage-associated lectin known as Mac-2 and also binds galectin 1. Antibodies for LGALS3BP are sold by Sigma-Aldrich, R&D Systems, and others.

Fc fragment of IgG binding protein (FCGBP; Gene ID: 8857; NCBI Reference Sequence: NM_{—}003890.2; also known as IgG Fc binding protein; FC(GAMMA)BP; Fcgamma-binding protein antigen; Human Fc gamma BP) has not been well characterized in the literature. This protein may be involved in the maintenance of the mucosal structure as a gel-like component of the mucosa. An antibody is sold by Sigma-Aldrich.

CD5 molecule-like (CD5L; Gene ID: 922; Swiss-Prot: 043866; NCBI Reference Sequence: NM_{—}005894.2; also known as API6; SP-alpha; apoptosis inhibitor 6; CD5 antigen-like; CT2) also is not well characterized in the scientific literature. It may play a role as an inhibitor of apoptosis and/or regulator of the immune system. Antibodies to this protein are sold by Sigma-Aldrich and R&D Systems.

Therapies for HCV-Infected Patients

Some aspects of the invention related to treatment of HCV in HCV-infected human subjects.

The current standard of care for HCV infection, pegylated interferon alpha in combination with ribavirin, has roughly 40% sustained viral response (SVR) for patients infected with genotype 1, which counts for 70% of chronic hepatitis C patients in developed countries, and 80% SVR in genotype 2 or 3 HCV-infected patients (McHutchison et al., N. Engl. J. Med., 339, 1485-1492 (1998); Davis et al., N. Engl. J. Med., 339, 1493-1499 (1998); McHutchinson et al., N. Engl. J. Med., 361, 580-539 (2009)).

RIBAVIRIN (CASRN: 36791-04-5; C₈—H₁₂—N₄—O₅; Mol. Wt. 244.20; 1-beta-D-Ribofuranosyl-1H-1,2,4-triazole-3-carboxamide) is a nucleoside analogue antiviral therapeutic that has been prepared and for oral administration (Capsules: 200 mg Rebetol (Schering); Tablets, film-coated: 200 mg Copegus (Roche)); nasal and oral inhalation (6 g Virazole (Valeant)); and topical administration. See, e.g., McEvoy, G. K. (ed.). American Hospital Formulary Service—Drug Information 2005. Bethesda, Md.: American Society of Health-System Pharmacists, Inc. 2005 (Plus Supplements), p. 811. Its structural formula is as follows:

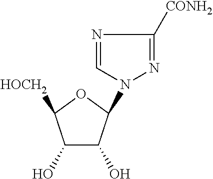

Ribavirin is used in combination with another therapeutic, e.g., an interferon, to treat subjects with HCV. Ribavirin treatment has been the subject of hundreds of studies and publications in the scientific literature. COPEGUS in combination with PEGASYS (peginterferon alfa-2a) is indicated for the treatment of adults with chronic hepatitis C (CHC) virus infection who have compensated liver disease and have not been previously treated with interferon alpha. The recommended dose of COPEGUS tablets is provided in the table below. The recommended duration of treatment for patients previously untreated with ribavirin and interferon is 24 to 48 weeks.

The daily dose of COPEGUS is 800 mg to 1200 mg administered orally in two divided doses. The dose should be individualized to the patient depending on baseline disease characteristics (e.g., genotype), response to therapy, and tolerability of the regimen. COPEGUS should be taken with food.

| PEGASYS and | | | |
|---|---|---|---|
| COPEGUS Dosing | | | |
| Recommendations | | | |
| Hepatitis C | PEGASYS | COPEGUS | |
| Virus (HCV) Genotype | Dose* | Dose | Duration |
| Genotypes 1, 4 | 180 mcg | <75 kg = 1000 mg | 48 weeks |
| | | ≧75 kg = 1200 mg | 48 weeks |
| Genotypes 2, 3 | 180 mcg | 800 mg | 24 weeks |

Suitable examples of interferon that can be employed in the invention include Albuferon (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); alpha-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C,” Hepatology, 33, pp. 433-438 (2001)); consensus interferon (INFERGEN®) (Kao, J. H., et al., “Efficacy of Consensus Interferon in the Treatment of Chronic Hepatitis,” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity,” Pathol. Biol. (Paris) 47, pp. 553-559 (1999)); and Omega Duros® delivering omega interferon via implantable Duros® (Intarcia Therapeutics, Inc., Mountain View, Calif.).

PEGASYS (Roche), peginterferon alfa-2a, is a covalent conjugate of recombinant alfa-2a interferon (approximate molecular weight 20,000 daltons) with a single branched bis-monomethoxy polyethylene glycol (PEG) chain (approximate MW 40,000 daltons). The PEG moiety is linked at a single site to the interferon-alfa moiety via a stable amide bond to lysine. Peginterferon alfa-2a has an approximate molecular weight of 60,000 daltons. Interferon alfa-2a is produced using recombinant DNA technology in which a cloned human leukocyte interferon gene is inserted into and expressed in Escherichia coli. PEGASYS has been tested in clinical trials as an HCV monotherapy as well as combination therapy with Ribavirin.

Telaprevir (VX-950) is a promising therapeutic under development by Vertex Pharmaceuticals Incorporated (Cambridge Mass.). VX-950 is described in International (PCT) Patent Publication Numbers WO 02/018369 and WO 2006/050250, and International Patent Application No. PCT/US2008/006572, filed on May 21, 2008, with reference to the following structural formula, or a pharmaceutically acceptable salt thereof:

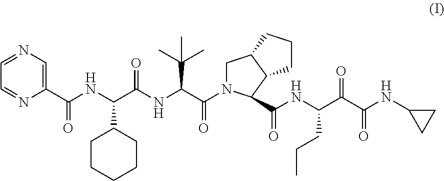 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

Additional description of VX-950 can be found in International Patent Publication Numbers WO 07/098,270 and WO 08/106,151. All of the foregoing applications and publications are hereby incorporated by reference in their entirety, and specifically for their teachings relating to VX-950.

VX-950 may be prepared in general by methods known to those skilled in the art (see, e.g., WO 02/18369). Any suitable formulations known in the art can be used in the invention. For example, formulations described in WO 2005/123075, WO 2007/109604, WO 2007/109605 and WO 2008/080167 can be employed in the invention. Other specific examples include:

| | | |
|---|---|---|
| VX-950 | 49.5 | wt % |
| HPMC 40 cp | 49.5 | wt % |
| SLS | 1 | wt % |
| VX-950 | 49.5 | wt % |
| HPC | 49.5 | wt % |
| SLS | 1 | wt % |
| VX-950 | 49.5 | wt % |
| PVP K30 | 49.5 | wt % |
| SLS | 1 | wt % |

VX-950 Solid Dispersion

| % (w/w) | Ingredient | |
|---|---|---|
| 49.5 | VX-950 | Spray-dried from a MeCl₂ solution |
| 49.5 | PVP K29/32 | |
| 1 | SLS | |

wherein HPMC (Hydroxypropyl Methylcellulose 60SH 50cP (Biddle Sawyer or Shin-Etsu Metolose, HPMC60SH50) (Hypromellose Acetate Succinate, HG grade, Shin-Etsu Chemical Co.), HPC (hydroxypropyl cellulose), PVP (polyvinylpyrrolidone) and SLS (Sodium Lauryl Sulfate) are as described in WO 2005/123075. In certain embodiments, the solid dispersion shown above can be suspended in a 1% HPMC, 0.002% simethicone solution (1 wt % HPMC, 0.002 wt % simethicone and 99 wt % water). Additional examples include 1:1 VX-950: PVPK30, 1 wt % SLS (Refreshed Tox.); Niro-49 wt % HPMCAS/1 wt % SLS/1 wt % SDBS/49% VX-950; 40.5 wt % PVP-VA/10 wt % ETPGS/49.5 wt % VX-950; 40.5 wt % HPMC/10 wt % ETPGS/49.5 wt % VX-950; 49 wt % VX950, 49 wt % HPMCAS, 1 wt % SLS, 1 wt % SDBS; and 49 wt % VX950, 16 wt % HPPh, 33 wt % HPC, 1 wt % SLS, wt % SDBS, wherein PVPK30 (Polyvinyl Pyrrolidone K30), SDBS (sodium dodecyl benzene sulfonate), HPMCAS (Hydroxypropyl Methylcellulose Acetate Succinate), Vitamin ETPGS, PVP (polyvinylpyrrolidone) and SLS (Sodium Lauryl Sulfate), and details of the preparation of these formulations can be found in WO 2005/123075.

Additional examples include those described in WO 2007/109604:
a solid dispersion comprising 55 wt % VX-950, 24.4 wt % HPMCAS-HG (Hydroxypropyl Methylcellulose Acetate Succinate, JPE (Biddle Sawyer or Shin-Etsu HPMCAS-HG grade), 19.6 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 55 wt % VX-950, 14.7 wt % HPMCAS-HG (Hydroxypropyl Methylcellulose Acetate Succinate, JPE (Biddle Sawyer or Shin-Etsu HPMCAS-HG grade), 29.3 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 60 wt % VX-950, 24.4 wt % HPMCAS-HG (Hydroxypropyl Methylcellulose Acetate Succinate, JPE (Biddle Sawyer or Shin-Etsu HPMCAS-HG grade), 14.6 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 65 wt % VX-950, 17 wt % HPMCAS-HG (Hydroxypropyl Methylcellulose Acetate Succinate, JPE (Biddle Sawyer or Shin-Etsu HPMCAS-HG grade), 17 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 70 wt % VX-950, 9.7 wt % HPMCAS-HG (Hydroxypropyl Methylcellulose Acetate Succinate, JPE (Biddle Sawyer or Shin-Etsu HPMCAS-HG grade), 19.3 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 60 wt % VX-950, 39 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS);
a solid dispersion comprising 49.5 wt % VX-950, 24.5 wt % HPMCAS-HG (Hydroxypropyl Methylcellulose Acetate Succinate, JPE (Biddle Sawyer or Shin-Etsu HPMCAS-HG grade), 24.5 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 83 wt % VX-950, 8 wt % HPMCAS-HG (Hydroxypropyl Methylcellulose Acetate Succinate, JPE (Biddle Sawyer or Shin-Etsu HPMCAS-HG grade), 8 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 49.5 wt % VX-950, 24.5 wt % HPMCAS-HG (Hydroxypropyl Methylcellulose Acetate Succinate, JPE (Biddle Sawyer or Shin-Etsu HPMCAS-HG grade), 24.5 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 70 wt % VX-950, 14.5 wt % HPMCAS-HG (Hydroxypropyl Methylcellulose Acetate Succinate, JPE (Biddle Sawyer or Shin-Etsu HPMCAS-HG grade), 14.5 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 65 wt % VX-950, 14.6 wt % HPMCAS-HG (Hydroxypropyl Methylcellulose Acetate Succinate, JPE (Biddle Sawyer or Shin-Etsu HPMCAS-HG grade), 19.4 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 65 wt % VX-950, 9.7 wt % HPMCAS-HG (Hydroxypropyl Methylcellulose Acetate Succinate, JPE (Biddle Sawyer or Shin-Etsu HPMCAS-HG grade), 24.3 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 60 wt % VX-950, 19.5 wt % HPMCAS-HG (Hydroxypropyl Methylcellulose Acetate Succinate, JPE (Biddle Sawyer or Shin-Etsu HPMCAS-HG grade), 19.5 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 60 wt % VX-950, 14.6 wt % HPMCAS-HG (Hydroxypropyl Methylcellulose Acetate Succinate, JPE (Biddle Sawyer or Shin-Etsu HPMCAS-HG grade), 24.4 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 70 wt % VX-950, 9.7 wt % HPMCAS-HG (Hydroxypropyl Methylcellulose Acetate Succinate, JPE (Biddle Sawyer or Shin-Etsu HPMCAS-HG grade), 19.3 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 49.5 wt % VX-950, 24.5 wt % HPMCAS-HG (Hydroxypropyl Methylcellulose Acetate Succinate, JPE (Biddle Sawyer or Shin-Etsu HPMCAS-HG grade), 24.5 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 83 wt % VX-950, 8 wt % HPMCAS-HG (Hydroxypropyl Methylcellulose Acetate Succinate, JPE (Biddle Sawyer or Shin-Etsu HPMCAS-HG grade), 8 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 49.5 wt % VX-950, 49.5 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS);

a solid dispersion comprising 83 wt % VX-950, 16 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS);
a solid dispersion comprising 82.44 wt % VX-950, 15.89 wt % HPMCAS-HG (Hydroxypropyl Methylcellulose Acetate Succinate, JPE (Biddle Sawyer or Shin-Etsu HPMCAS-HG grade), and 1.67 wt % Sodium Lauryl Sulfate (SLS);
a solid dispersion comprising 49.5 wt % VX-950, 24.75 wt % HPMCAS-HG (Hydroxypropyl Methylcellulose Acetate Succinate, JPE (Biddle Sawyer or Shin-Etsu HPMCAS-HG grade), 24.75 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS).
a solid dispersion comprising 60 wt % VX-950, 24.6 wt % HPMCAS-HG (Hydroxypropyl Methylcellulose Acetate Succinate, JPE (Biddle Sawyer or Shin-Etsu HPMCAS-HG grade), 14.4 wt % HPMC-60SH (Hydroxypropyl Methylcellulose 60SH 50cP (Biddle Sawyer or Shin-Etsu Metolose, HPMC60SH50), and 1 wt % Sodium Lauryl Sulfate (SLS);
a solid dispersion comprising 60 wt % VX-950, 39 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS); and
a solid dispersion comprising 49.5 wt % VX-950, 49.5 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS).

Details of the preparation of these solid dispersions are described in WO 2007/109604.

Additional specific examples include tablet formulations containing a spray dried dispersion of VX-950, which are described in WO 2007/109604:

| Component | mg per | |
|---|---|---|
| Roller compaction blend | Tablet | Percent |
| VX950 Spray Dried Dispersion1 | 505.1 | 74.9 |
| Pharmatose DCL 22 (Lactose, USP/NF, PhEur, [symbol] | 37.5 | 5.6 |
| Ac-Di-Sol (cross carmellose sodium, NF, PhEur, | 24.0 | 3.6 |
| Extragranular addition | | 0.0 |
| Avicel pH 113 | 33.7 | 5.0 |
| Vitamin E TPGS (NF) | 24.0 | 3.6 |
| Ac-Di-Sol (cross carmellose sodium, NF, PhEur, | 16.0 | 2.4 |
| Cabosil M-5 (colloidal silicon dioxide, NF, PhEur) | 8.0 | 1.2 |
| Sodium Stearyl fumarate (NF, PhEur, JP) | 26.0 | 3.9 |
| Total Formulation weight | 674.3 | 100.0 |

[symbol]

indicates data missing or illegible when filed

Additional specific examples include tablet formulations described in WO2008/080167:

VX950 SD Tableting Experiment
Design (Potency: 250 mg VX950)

| Trial # | Vit E type | Vit E type |
|---|---|---|
| A | VitE-TPGS (24 mg) | Granulated VitE on excipients |
| C | VitE- Acetate (48 mg) | Used as is |
| E | Vit E-TPGS (24 mg) | Vit E Spray Congealed |
| F | Vit E-TPGS (24 mg) | Granulated Vit E onto VX950 |

Trial# A Formulation

| | Ingredients | Wt/Tablet | |
|---|---|---|---|
| Item | Physical mixture | (mg) | wt % |
| 1 | Solid Dispersion | 339.9 | 66.32 |
| | (73.55% VX950/26.45% HPMCAS) | | |
| 2 | PHARMATOSE ® DCL 22 (Lactose) | 37.5 | 7.32 |
| 3 | AC-DI-SOL ® (Cross carmellose sodium) | 24.0 | 4.68 |
| 4 | Sodium Stearyl Fumarate | 1.6 | 0.32 |
| 5 | SLS | 3.4 | 0.66 |
| 6 | AVICEL ® pH 113 | 33.7 | 6.58 |
| | (Microcrystalline cellulo [symbol] | | |
| 7 | Vitamin E TPGS (granulated on excipients) | 24.0 | 4.68 |
| 8 | AC-DI-SOL ® (Cross carmellose sodium) | 16.0 | 3.12 |
| 9 | Cabosil M-5 (Colloidal silicon dioxide) | 8.0 | 1.56 |
| 10 | Sodium Stearyl Fumarate | 24.4 | 4.76 |
| | Total | 512.5 | 100 |

Note:
VX 950 SD Lot 02
Potency: 250 mg VX950 [symbol]
indicates data missing or illegible when filed

Trial# C Formulation

| | Ingredients | Wt/Tablet | |
|---|---|---|---|
| Item | Physical mixture | (mg) | wt % |
| 1 | Solid Dispersion | 339.9 | 63.36 |
| | (73.55% VX950/26.45% HPMCAS) | | |
| 2 | PHARMATOSE ® DCL 22 (Lactose) | 37.5 | 6.99 |
| 3 | AC-DI-SOL ® (Cross carmellose sodium) | 24.0 | 4.47 |
| 4 | Sodium Stearyl Fumarate | 1.6 | 0.30 |
| 5 | SLS | 3.4 | 0.63 |
| 6 | AVICEL ® pH 113 | 33.7 | 6.28 |
| | (Microcrystalline cellulose) | | |
| 7 | Vitamin E-Acetate | 48.0 | 8.95 |
| 8 | AC-DI-SOL ® (Cross carmellose sodium) | 16.0 | 2.98 |
| 9 | Cabosil M-5 (Colloidal silicon dioxide) | 8.0 | 1.49 |
| 10 | Sodium Stearyl Fumarate | 24.4 | 4.54 |
| | Total | 536.5 | 100 |

Trial# E Formulation

| | Ingredients | Wt/Tablet | |
|---|---|---|---|
| Item | Physical mixture | (mg) | wt % |
| 1 | Solid Dispersion | 339.9 | 66.32 |
| | (73.55% VX950/26.45% HPMCAS) | | |
| 2 | PHARMATOSE ® DCL 22 (Lactose) | 37.5 | 7.32 |
| 3 | AC-DI-SOL ® (Cross carmellose sodium) | 24.0 | 4.68 |
| 4 | Sodium Stearyl Fumarate | 1.6 | 0.32 |
| 5 | SLS | 3.4 | 0.66 |
| 6 | AVICEL ® pH 113 | 33.7 | 6.58 |
| | (Microcrystalline cellulose) | | |
| 7 | Vitamin E Spray Congealed | 24.0 | 4.68 |
| 8 | AC-DI-SOL ® (Cross carmellose sodium) | 16.0 | 3.12 |
| 9 | Cabosil M-5 (Colloidal silicon dioxide) | 8.0 | 1.56 |
| 10 | Sodium Stearyl Fumarate | 24.4 | 4.76 |
| | Total | 512.5 | 100 |

Note:
VX 950 SD Lot 02
Potency: 250 mg VX950

Trial# F Formulation

| | | Wt/Tablet | |
|---|---|---|---|
| Item | Ingredients | (mg) | wt % |
| 1 | Solid Dispersion | 339.9 | 66.32 |
| | (73.55% VX950/26.45% HPMCAS) | | |
| 2 | Vitamin E granulated onto dispersion | 24.0 | 4.68 |
| 3 | PHARMATOSE ® DCL 22 (Lactose) | 37.5 | 7.32 |
| 4 | AC-DI-SOL ® (Cross carmellose sodium) | 24.0 | 4.68 |
| 5 | Sodium Stearyl Fumarate | 1.6 | 0.32 |
| 6 | SLS | 3.4 | 0.66 |
| 7 | AVICEL ® pH 113 | 33.7 | 6.58 |
| | (Microcrystalline cellulose) | | |
| 8 | AC-DI-SOL ® (Cross carmellose sodium) | 16.0 | 3.12 |
| 9 | Cabosil M-5 (Colloidal silicon dioxide) | 8.0 | 1.56 |
| 10 | Sodium Stearyl Fumarate | 24.4 | 4.76 |
| | Total | 512.5 | 100 |

Note:
VX 950 SD Lot 02
Potency: 250 mg VX950

Methods of the invention may be used to evaluate likelihood of successful therapy with respect to any of these therapeutics, alone or in combination, as well as other therapeutics for hepatitis infections, especially HCV.

Formulation and Dosing Considerations for Therapeutics

Generally in the invention, “administration” or “co-administration” of one or more therapeutic agents (including VX-950, interferon, ribavirin, and any combination thereof) includes administering each active therapeutic agent in the same dosage form or in different dosage forms. When administered in different dosage forms, the active therapeutic agent may be administered at different times, including simultaneously or in any time period around administration of the other dosage forms. Separate dosage forms may be administered in any order. That is, any dosage forms may be administered prior to, together with, or following the other dosage forms.

VX-950 and any additional agent may be formulated in separate dosage forms. Alternatively, to decrease the number of dosage forms administered to a patient, VX-950 and any additional agent may be formulated together in any combination. Any separate dosage forms may be administered at the same time or different times. It should be understood that dosage forms should be administered within a time period such that the biological effects were advantageous.

For approved therapeutics, preferred doses and dosage forms include those specified in the manufacturer's label, and additional dosing regimen that become adopted by practitioners in the field.

If pharmaceutically acceptable salts are employed in the invention as active therapeutic agents, those salts are typically derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates; long chain halides such as decyl, lauryl, myristyl and stearyl chlorides, bromides and iodides; aralkyl halides, such as benzyl and phenethyl bromides; and others. Water or oil-soluble or dispersible products are thereby obtained.

In the invention, as desired, modification of therapeutic agent(s) can also be employed by, for example, appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Typically, one or more therapeutic agents, including VX-950 and interferon, employed in the invention are included in pharmaceutical compositions, though the therapeutic agent(s) may be administered alone. A “pharmaceutical composition” means a composition comprising a therapeutic agent disclosed herein, and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms. The compositions may be presented in the form of tablets, pills, granules, powders, aqueous solutions or suspensions, injectable solutions, elixirs or syrups.

Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monosterate and gelatin. Exemplary adsorption promoting agents to enhance absorption include dimethyl sulphoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween 60, Tween 80, cetostearyl alcohol, myristyl alcohol, glyceryl mono-stearate and sodium lauryl sulfate, fatty acid esters of sorbitan, vegetable oils (such as cottonseed oil, groundnut oil, corn germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate phosphate. Exemplary disintegrating agents include starch, alginic acids and certain complex silicates. Exemplary lubricants include magnesium stearate, sodium lauryl sulphate, talc, as well as high molecular weight polyethylene glycols.

The choice of material in the pharmaceutical composition other than the therapeutic agent is generally determined in accordance with the chemical properties of the therapeutic agent, such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the therapeutic agent to be administered to the patient is in liquid form, for example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e., an alcohol having two or more hydroxyl groups, such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired cosmetic properties. Thus the cream should preferably be a non-greasy, non-staining and washable product with suitable consistency to avoid leakage from tubes or other containers.

Straight or branched chain, mono- or di-basic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Generally, a therapeutic agent/pharmaceutical compositions disclosed herein may be administered in a suitable formulation to humans and animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of a therapeutic agent (including VX-950) disclosed herein, and includes, for example, tablets, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Moulded tablets may be made by moulding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, a therapeutic agent disclosed herein can be microencapsulated in, or attached to, a slow release or targeted delivery systems such as a biocompatible, biodegradable polymer matrices (e.g., poly (d,l-lactide co-glycolide)), liposomes, and microspheres and subcutaneously or intramuscularly injected by a technique called subcutaneous or intramuscular depot to provide continuous slow release of the compound (s) for a period of 2 weeks or longer. The therapeutic agent may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for nasal or inhalational administration” means formulations which are in a form suitable to be administered nasally or by inhalation to a patient. The formulation may contain a carrier, in a powder form, having a particle size for example in the range 1 to 500 microns (including particle sizes in a range between 20 and 500 microns in increments of 5 microns such as 30 microns, 35 microns, etc.). Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalational therapy is readily administered by metered dose inhalers.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The therapeutic agent may also be presented as a bolus, electuary or paste.

“Formulations suitable for parenteral administration” means formulations that are in a form suitable to be administered parenterally to a patient. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for rectal or vaginal administrations” means formulations that are in a form suitable to be administered rectally or vaginally to a patient. The formulation is preferably in the form of suppositories that can be prepared by mixing the compounds of this invention with suitable non-irritating excipients or carriers such as cocoa butter, polyethylene glycol or a suppository wax, which are solid at ordinary temperatures but liquid at body temperature and, therefore, melt in the rectum or vaginal cavity and release the active component.

“Formulations suitable for systemic administration” means formulations that are in a form suitable to be administered systemically to a patient. The formulation is preferably administered by injection, including transmuscular, intravenous, intraperitoneal, and subcutaneous. For injection, the compounds of the invention are formulated in liquid solutions, preferably in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of a therapeutic agent disclosed herein is solid form, for example capsules, tablets, pills, powders, dragees or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl, (j) opacifying agents, (k) buffering agents, and agents which release the compound(s) of the invention in a certain part of the intestinal tract in a delayed manner.

The amount of active therapeutic agent(s) that may be combined with the carrier and/or excipient materials to produce a single dosage form will vary depending upon the host treated and the particular mode of administration. A typical preparation will contain from about 5% to about 95% active therapeutic agent (w/w). Preferably, such preparations contain from about 20% to about 80% therapeutic agent.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The pharmaceutical compositions and dosage formulations disclosed herein are preferably for use in vivo. Nevertheless, this is not intended as a limitation to using of the pharmaceutical compositions and dosage formulations for any purpose. For example, a biological substance pre-treated with a pharmaceutical composition disclosed herein can also be employed in the invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

It should also be understood that a specific dosage and treatment regimen for any particular patient will depend upon a variety of factors, including the activity of the specific compound employed, the age, body weight, general health, sex, diet, time of administration, rate of excretion, drug combination, the judgment of the treating physician and the severity of the particular disease being treated, prior treatment history, co-morbidities or concomitant medications, baseline viral load, race, duration of diseases, status of liver function and degree of liver fibrosis/cirrhosis, and the goal of therapy (eliminating circulating virus per-transplant or viral eradication). The amount of active ingredients will also depend upon the particular described compound and the presence or absence and the nature of the additional anti-viral agent in the composition.

Measuring of Biomarkers

Any technique that is available for quantifying a protein may be used for quantifying proteins in the context of methods of the invention. For example, quantitative mass spectrometry techniques exist and are suitable for measuring protein in a sample, including measuring small amounts of protein in a small sample. Numerous antibody-based methods exist for quantifying proteins in samples, including Western blot techniques and ELISA assays. For proteins with enzymatic or other activities, activity assays, such as enzymatic activity assays using a substrate, provide a measure of the amount of active protein in a sample.

In some preferred variations, protein biomarkers are identified and/or quantified with an immunoassay, using one or more antibodies that preferentially bind, and preferably bind with high specificity, to a biomarker of interest. Exemplary immunoassays include immunofluorescent immunoassays, immunoprecipitations, radioimmunoasays, ELISA, and Western blotting. The epitope(s) used for recognizing and quantifying a marker may be a linear peptide epitope, a conformational epitope, an epitope that includes one or more side-chain modifications (e.g., glycosylation), and so on. See generally E. Maggio, Enzyme-Immunoassay, (1980) (CRC Press, Inc., Boca Raton, Fla.); see also U.S. Pat. No. 4,727,022 to Skold et al. titled “Methods for Modulating Ligand-Receptor Interactions and their Application,” U.S. Pat. No. 4,659,678 to Forrest et al. titled “Immunoassay of Antigens,” U.S. Pat. No. 4,376,110 to David et al., titled “Immunometric Assays Using Monoclonal Antibodies,” U.S. Pat. No. 4,275,149 to Litman et al., titled “Macromolecular Environment Control in Specific Receptor Assays,” U.S. Pat. No. 4,233,402 to Maggio et al., titled “Reagents and Method Employing Channeling,” and U.S. Pat. No. 4,230,767 to Boguslaski et al., titled “Heterogeneous Specific Binding Assay Employing a Coenzyme as Label.”

Antibodies

The term “antibody” refers to a complete (intact) antibody (immunoglobulin) molecule (including polyclonal, monoclonal, chimeric, humanized, or human versions having full length heavy and/or light chains) or an antigen-binding fragment thereof. Antibody fragments include F(ab′)₂, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see, e.g., Hollinger and Hudson, Nature Biotechnology, 23(9), 1126-1136 (2005)). Antibody polypeptides, including monobodies, also are disclosed in U.S. Pat. No. 6,703,199. Other antibody polypeptides are disclosed in U.S. Patent Publication No. 2005-0238646. Conventional monoclonal and polyclonal antibodies are suitable for many biomarker assays. In some preferred variations, the antibody may optionally further comprise a label, such as a fluorescent, enzymatic, or radioactive label.

The term “specifically binds” refers to the ability of the antibody or fragment thereof to bind to a target antigen, e.g., as it exists in a biological sample such as blood, serum, or plasma, with greater affinity (e.g., preferably at least 10, 15, 20, 25, 50, 100, 250, 500, 1000, or 10,000 times greater affinity) than it binds to other components/proteins that may be found in the biological sample. Generally speaking, greater affinity, avidity, and specificity permits more accurate measurement of target proteins.

If available, commercial antibodies directed to biomarkers of the invention are expected to be suitable for measuring the biomarkers. However, many procedures are known within the art for producing antibodies, any of which are suitable for production an antibody against a biomarker. The antibody or antibody fragment can be isolated from an immunized animal, synthetically made, or genetically-engineered. Antibodies to a biomarker protein can be obtained, for example, by immunizing an animal with the protein, polypeptide, or fragment thereof, or by introducing into an animal an expression vector encoding the biomarker or fragment thereof to achieve protein production in vivo. Prior to administration in some instances, a peptide immunogen is covalent coupled to another immunogenic protein, for example, a carrier protein such as keyhole limpet hemocyanin (KLH) or bovine serum albumin (BSA), and/or combined with an adjuvant, such as Freund's complete or incomplete adjuvant. Polyclonal antibodies are typically raised in non-human animals such as rats, mice, rabbits, goats, cattle, or sheep, and also can be raised in a subhuman primate as described in, e.g., International Patent Publication WO 1991/11465 and Losman et al., Int. J. Cancer, 46, 310 (1990). Antibodies also can be selected by screening peptide or antibody libraries for high affinity binding molecules.

An antibody or fragment thereof also can be genetically-engineered such that the antibody or antibody fragment comprises, e.g., a variable region domain generated by recombinant DNA engineering techniques. For example, a specific antibody variable region can be modified by insertions, deletions, or changes in the amino acid sequence of the antibody to produce an antibody of interest. In this regard, polynucleotides encoding complementarity determining regions (CDRs) of interest are prepared, for example, by using polymerase chain reaction to synthesize variable regions using mRNA of antibody-producing cells as a template (see, for example, Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley-Liss, Inc. 1995); and Larrick et al., Methods: A Companion to Methods in Enzymology, 2, 106-110 (1991)). Antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target.

Monoclonal antibodies are generated using a variety of techniques, such as those known in the art (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). Typically, monoclonal antibodies are produced by a hybridoma, and the invention provides a hybridoma that produces the inventive monoclonal antibody or antibody fragment. Production of antibodies via immunization of non-human mammals and production of monoclonal antibodies is further described in, e.g., U.S. Pat. No. 7,381,409.

Antibody fragments derived from an intact antibody can be obtained, e.g., by proteolytic hydrolysis of the antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244 (1960); Porter, Biochem. J., 73: 119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1-2.8.10 and 2.10A.1-2.10A.5. Alternatively, such fragments may also be generated by recombinant genetic engineering techniques, such as those techniques known in the art and described herein.

Hybridization Assays

In variations that involve the measuring of mRNA that encodes a protein biomarker, any quantitative nucleic acid assay may be used. For example, many quantitative hybridization and polymerase chain reaction procedures exist for quantitatively measuring the amount of an mRNA transcript in a biological sample. (See, e.g., Current Protocols in Molecular Biology, Ausubel et al., eds., John Wiley & Sons (2007), including all supplements.) Selection of one or more suitable probes that are specific for an mRNA, and selection of hybridization or PCR conditions, are within the ordinary skill of scientists who work with nucleic acids.

Enzymatic Assays

For biomarkers of interest that are enzymatic (e.g., CNDP1, a dipeptidase), measurements of enzymatic activity may be used as a surrogate for measurement of the biomarker. In a typical enzymatic activity assay, the biological sample or fraction thereof is contacted with a substrate for the enzyme under conditions suitable for enzymatic activity, and product of the enzymatic reaction is measured over time. For example, in some variations, a catalytic activity of CNDP1 involves preferential hydrolysis of the beta-Ala-|-H is dipeptide (carnosine), and also anserine, Xaa-|-His dipeptides and other dipeptides including homocarnosine. In some variations, one measures the cleavage of this dipeptidase over time in the samples relative to controls to determine the amount of enzyme present. See, e.g., Teufel et al., “Sequence Identification and Characterization of Human Carnosinase and a Closely Related Non-specific Dipeptidase,” J. Biol. Chem. 278(8): 6521-31 (2003), incorporated herein by reference.

Kits

An aspect of the invention is kits that contain reagents useful for measuring combinations of biomarkers taught in the invention. For example, the kit may contain, in separate containers but packaged together, an antibody specific for each biomarker of interest. In some variations, the antibody is pre-bound to a solid matrix such as a plate or bead. In other variations, the kit may include reagents to attach an antibody to a solid matrix. Optionally, the kit further includes positive and/or negative control formulations for each biomarker to be screened. Optionally, the kit further includes one or more detectable labels and/or secondary antibodies for quantifying binding between the primary antibody and the biomarker.

Computer-Related Aspects of the Invention

A machine-readable storage medium can comprise a data storage material encoded with machine readable data or data arrays which, when using a machine programmed with instructions for using said data, is capable of use for a variety of purposes, such as, evaluating subjects for treatment, such as evaluating the likelihood that a particular HCV treatment will be effective to achieve a sustained viral response or cure in an HCV-infected subject. Measurements of biomarkers and/or the resulting evaluation of therapeutic efficacy from those biomarker measurements can implemented in computer programs executing on programmable computers, comprising, inter alia, a processor, a data storage system (including volatile and non-volatile memory and/or storage elements), at least one input device, and at least one output device. Program code can be applied to input data to perform the functions described above and generate output information. The output information can be applied to one or more output devices, according to methods known in the art. The computer may be, for example, a personal computer, microcomputer, or workstation of conventional design.

Each program can be implemented in a high level procedural or object oriented programming language to communicate with a computer system. However, the programs can be implemented in assembly or machine language, if desired. The language can be a compiled or interpreted language. Each such computer program can be stored on a storage media or device (e.g., ROM or magnetic diskette or others as defined elsewhere in this disclosure) readable by a general or special purpose programmable computer, for configuring and operating the computer when the storage media or device is read by the computer to perform the procedures described herein.

Levels of an biomarkers that correlate with therapeutic outcomes can then be determined and compared to a reference value, e.g. a control subject or population whose biomarker measurements and response to a particular therapy are known, or an index value or baseline value. The reference sample or index value or baseline value may be taken or derived from one or more subjects who have been exposed to the treatment, for example, and followed to determine the efficacy of the treatment. A reference value can also comprise a value derived from prediction algorithms or computed indices from population studies such as those disclosed herein.

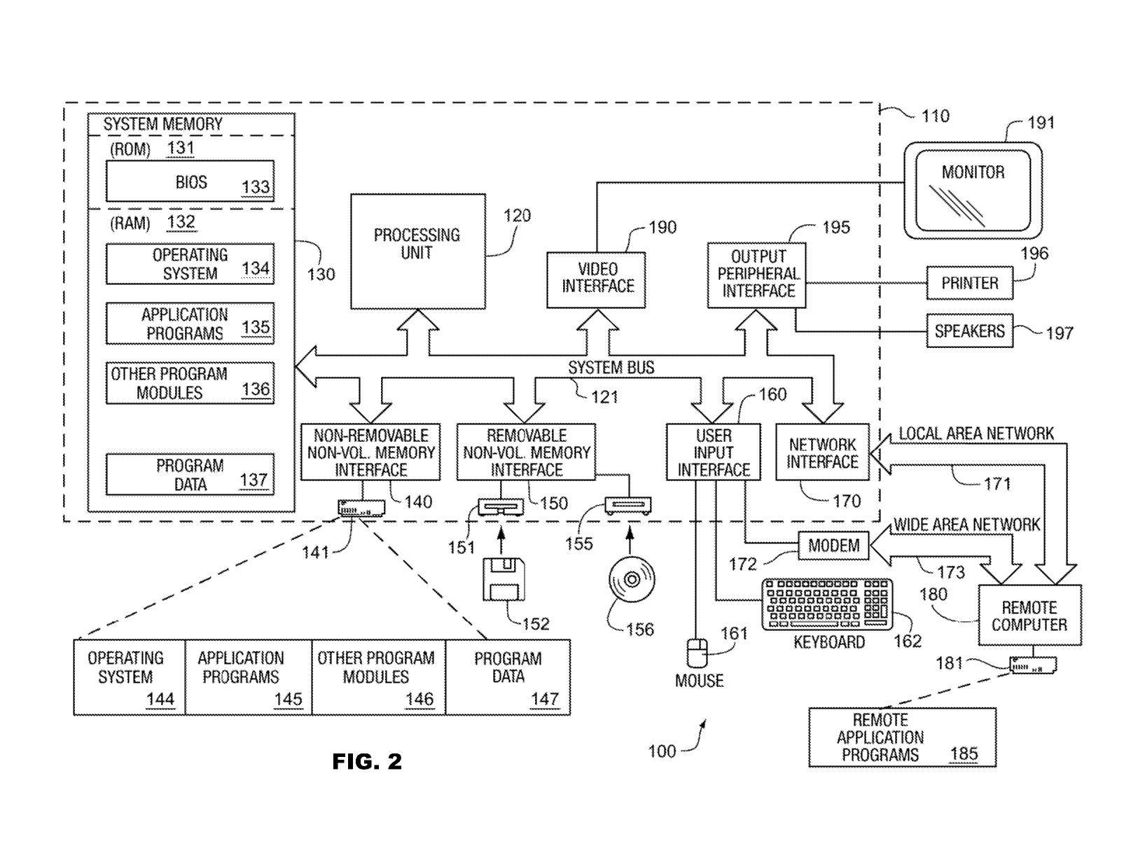

FIG. 2 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method of apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like, including those systems, environments, configurations and means described elsewhere within this disclosure.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 2, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 2 illustrates operating system 134, application programs 135, other program modules 136, and program data 137.

The computer 110 may also include other removable/non-removable, volatile/nonvolatile computer storage media. By way of example only, FIG. 2 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 2, provide storage of computer readable instructions, data structures, program modules and other data for the computer 110. In FIG. 2, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The biomarkers of the present invention can thus be used to generate a biomarker profile of those subjects with HCV infection who respond to a particular HCV treatment; and also generate a biomarker profile of subjects who do not respond to the treatment. In some variations, the subjects in the two groups are matched for one or more clinical parameters to optimize the value of the biomarkers for subjects who have similar parameters. A test subject's biomarker profile can be compared to a reference biomarker profile to evaluate whether the test subject is likely to benefit from a particular treatment. The biomarker profiles of the present invention can be contained in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory test factors described herein and/or known to clinicians. Alternatively or additionally, the machine-readable media can also comprise subject information such as medical history and any relevant family history. The machine-readable media can also contain information relating to other algorithms and computed indices such as those described herein.

Summary of Algorithm Development Process and Application of Algorithms

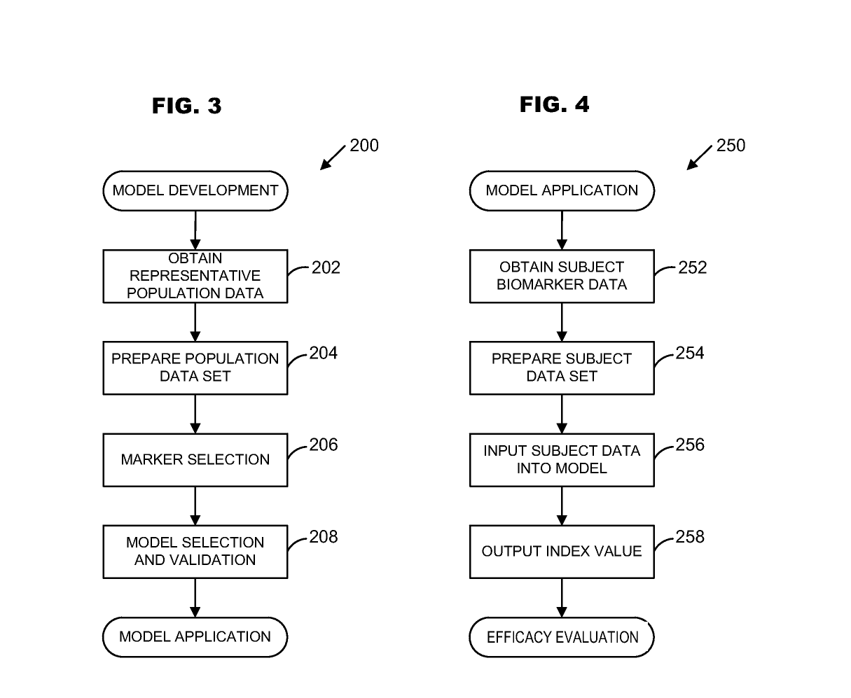

FIG. 3 is a flow diagram of an example method 200 for developing a model which may be used to evaluate a likelihood of a person, or group of people, infected with HCV for responding favorably (achieving sustained viral response or cure) to an HCV treatment. The method 200 may be implemented using the example computing system environment 100 of FIG. 2 and will be used to explain the operation of the environment 100. However, it should be recognized that the method 200 could be implemented by a system different than the computing system environment 100. At a block 202, biomarker data from a representative population, as has been described herein, is obtained from a data storage device, such as the system memory 130, an internal or external database, or other computer storage media. The biomarker data may be initially derived through a variety of means, including prospective (longitudinal) studies to involving observations of the representative population over a period of time, retrospective studies of samples of a representative population that queries the samples and/or from a retrospective epidemiological data storage containing the results from previous studies, such as an NIH database. The biomarker data may be derived from a single study or multiple studies, and generally includes data pertaining to the desired indication and endpoint of the representative population, including values of the biomarkers described herein, clinical annotations (which may include endpoints), and endpoints across many subjects.

At a block 204, the representative population data set is prepared as needed to meet the requirements of the model or analysis that will be used for biomarker selection, as described below. For example, data set preparation may include preparing the biomarker values from each subject within the representative population, or a chosen subset thereof. However, the raw biomarker data alone may not be entirely useful for the purposes of model training. As such, various data preparation methods may be used to prepare the data, such as gap fill techniques (e.g., nearest neighbor interpolation or other pattern recognition), quality checks, data combination using of various formulae (e.g., statistical classification algorithms), normalization and/or transformations, such as logarithmic functions to change the distribution of data to meet model requirements (e.g., base 10, natural log, etc.). Again, the particular data preparation procedures are dependent upon the model or models that will be trained using the representative population data. The particular data preparation techniques for various different model types are known, and need not be described further.

At a block 206, the particular biomarkers are selected to be subsequently used in the training of the model used to evaluate likelihood of success for a particular treatment regimen. Biomarker selection may involve utilizing a selection model to validate the representative population data set and selecting the biomarker data from the data set that provides the most reproducible results. Examples of data set validation may include, but are not limited to, cross-validation and bootstrapping. From the marker selection, the model to be used in evaluating a likelihood of favorable response to an HCV treatment may be determined and selected. However, it is noted that not all models provide the same results with the same data set. For example, different models may utilize different numbers of biomarkers and produce different results, thereby adding significance to the combination of biomarkers on the selected model. Accordingly, multiple selection models may be chosen and utilized with the representative population data set, or subsets of the data set, in order to identify the optimal model for evaluating a treatment protocol for a particular subject. Examples of the particular models, including statistical models, algorithms, etc., which may be used for selecting the biomarkers are described herein, and others are known in the art.

For each selection model used with the data set, or subset thereof, the biomarkers are selected based on each biomarker's statistical significance in the model. When input to each model, the biomarkers are selected based on various criteria for statistical significance, and may further involve cumulative voting and weighting. Tests for statistical significance may include exit-tests and analysis of variance (ANOVA). The model may include classification models (e.g., LDA, logistic regression, SVM, RF, tree models, etc.) and survival models (e.g., cox), many examples of which have been described above.

It is noted that while biomarkers may be applied individually to each selection model to identify the statistically significant biomarkers, in some instances individual biomarkers alone may not be fully indicative of the likelihood that a treatment will be successful, in which case combinations of biomarkers may be applied to the selection model. For example, rather than utilizing univariate biomarker selection, multivariate biomarker selection may be utilized. That is, a biomarker may not be a good indicator when used as a univariate input to the selection model, but may be a good indicator when used in combination with other biomarkers (i.e., a multivariate input to the model), because each marker may bring additional information to the combination that would not be indicative if taken alone.

At a block 208, the model to be used for evaluating likelihood of successful treatment is selected, trained and validated. In particular, leading candidate models may be selected based on one or more performance criteria, examples of which have been described above. For example, from using the data set, or data subsets, with various models, not only are the models used to determine statistically significant biomarkers, but the results may be used to select the optimal models along with the biomarkers. As such, the evaluation model used to evaluate likelihood of therapeutic efficacy may include one of those used as a selection model, including classification models and survival models. Combinations of models markers, including marker subsets, may be compared and validated in subsets and individual data sets. The comparison and validation may be repeated many times to train and validate the model and to choose an appropriate model, which is then used as an evaluation model for evaluating likelihood of success of an HCV therapy.

FIG. 4 is a flow diagram of an example method 250 for using a model to evaluate likelihood that an HCV-infected subject (e.g., a person, or group of people) will benefit from an HCV treatment regimen. At a block 252, biomarker data from the subject is obtained from a data storage device, which may be the same as, or different from, the data storage device discussed above with reference to FIG. 3. The subject biomarker data may be initially derived through a variety of means, including measuring from one or more biological samples; self-reports; physical examination; laboratory testing; existing medical records, charts or databases; and combinations thereof. As with the representative population biomarker data at block 204 of FIG. 3, the subject biomarker data at block 254 may be prepared using transforms, logs, combinations, normalization, etc. as needed according to the model type selected and trained in FIG. 3. Once the data has been prepared, at a block 256, the subject biomarker data is input into the evaluation model, and at a block 258 the evaluation model outputs an index value (e.g., probability of successful therapy, or score/grade representative of such probability, etc.).

In some cases, the model include or use a “predetermined criterion” based on measurements of the marker(s) in HCV-infected subjects who have received a therapy and whose results from the therapy has been tracked. For example, with respect to a single marker model, a subject's marker measurement could be compared to a measure of the same marker obtained from samples from a plurality of subjects. In some variations, the predetermined criterion is a measure of the marker in subjects that all achieved sustained viral response or cure, in which case marker measurement in subjects likely to benefit is comparable to the predetermined criteria. In other variations, the predetermined criterion is a measure of the marker in HCV patients who did not achieve the desired successful response to treatment, in which case the measurement in subjects likely to benefit will be significantly different. The model may include information such as mean, standard deviation, quartile measurements, confidence intervals, or other information about the distribution or range of marker concentration and predictive value with respect to clinical outcome. In still other variations, the predetermined criterion is a receiver operating characteristic curve based on data of marker measurements in HCV subjects who achieved a desired therapeutic endpoint and HCV-infected subjects who did not. In still other variations, the predetermined criterion is a cutoff value of marker concentration, wherein the cutoff value is determined, based on previous measurements to discriminate successful treatment with a sensitivity and specificity calculated from measurements of the marker in a population as described herein. Optionally, the predetermined criterion is based on subjects further stratified by other characteristics, e.g., clinical parameters, that can be determined for a subject.

One simple method for converting single protein measurements into a probability score for likelihood of success of a therapy (e.g., standard-of-care or telaprevir) is as follows:
a. Identify the expected success rate of the therapy for the patient population at large (e.g., either all HCV-infected patients, or a subset of HCV-infected patients that have been further stratified by other criteria); approximately 75% of telaprevir/SOC therapy achieve SVR based on results of Vertex phase III studies in treatment-naïve patients; about 45% of subjects receiving SOC alone achieve SVR based on historical data;
b. For each protein biomarker for which higher levels correlate with a better response, identify the numeric level of that protein such that the percentage of subjects identified in step 1 have a lower level of that protein; for each protein biomarker for which lower levels correlate with a better response, identify the numeric level of that protein such that the percentage of subjects identified in step 1 have a higher level of that protein.
c. For each protein biomarker for which higher levels of the protein correlate with better response, patients with levels greater than the level identified in step b are identified as patients with the best chance to respond to the therapy; for each protein biomarker for which lower levels correlate with a better response, patients with levels at or less lower than the level identified in step b are identified as patients with the best chance to respond to therapy.
   These predictions can be converted to numeric likelihood of success on a protein-by-protein basis by analyzing the raw data.

The invention is further illustrated and described with reference to the following Examples.

### EXAMPLE 1

A total of 172 plasma samples derived from patients in three different clinical trials of Telaprevir for the treatment of hepatitis C were analyzed. A goal of this study is to identify predictive markers associated with sustained viral response (SVR) to Telaprevir. The study also revealed useful data regarding predictive markers for current standard of care (SOC) therapy involving PEG-interferon plus Ribavirin.

A total of 8374 components were tracked across all samples. Of these, 1350 were found to be statistically differentially expressed in at least one of the comparisons performed. Following sequencing of the components, peptides were identified that cluster into 66 differentially expressed proteins. The greatest number (34) of statistically differentially expressed proteins correspond to the comparison between SVR-positive and SVR-negative patients treated with the SOC plus Telaprevir. Expression levels of ten proteins distinguished SOC-treated responders and non-responders. Half of these correspond to (overlap with) the Telaprevir comparison, and half of the SOC markers are specific to SOC alone.

Proteins were also found that could separate Telaprevir from SOC, but only when comparing between trials 1 & 2 and trial 3. Additional studies will be helpful to determine whether other variables (such as differences in sample collection between the trials) contributed to the results observed in those comparisons.

Nearly all proteins identified in this study are derived from liver, and most of those are known to be released into the blood. Thus, the results are highly consistent with HCV infection, and many of the candidate biomarkers have biological associations with liver function or liver damage.

### Scope of Study

Proteomic analysis of human plasma from three clinical trials of Telaprevir (VX-950) for the treatment of hepatitis C virus.

### Study Objectives

Objectives of the study include
1. Identify predictive biomarkers of SVR or non-response in patients treated with SOC plus Telaprevir.
2. Identify predictive biomarkers of SVR or non-response in patients treated with SOC.
3. Distinguish between SVR positive patients receiving SOC alone as compared with SOC plus Telaprevir.
4. Identify predictive biomarkers of response to treatment.

### Study Design

Samples used in this study were from patients participating in three separate clinical trials and were collected prior to treatment initiation. All patients received either the SOC or the SOC plus Telaprevir. Samples were segregated into five main treatment/response groups, A to E, as described in Table 1. African American patients were evaluated separately due to previously observed differential response rates to therapy in this population.

TABLE 1
Study samples

| | | | | | Number |
|---|---|---|---|---|---|
| | | | Clinical | | of |
| Group | Treatment | Race | Study | Response | samples |
| A | SOC | Caucasian | PROVE 1 & 2 | SVR | 25 |
| | | | | negative | |
| B | SOC | Caucasian | PROVE 1 & 2 | SVR | 25 |
| | | | | positive | |
| C1 | SOC + | Caucasian | PROVE 1 & 2 | SVR | 9 |
| | Telaprevir | | | negative | |
| C2 | SOC + | Caucasian | PROVE 3 | SVR | 29 |
| | Telaprevir | | | negative | |
| D1 | SOC + | Caucasian | PROVE 1 & 2 | SVR | 20 |
| | Telaprevir | | | positive | |
| D2 | SOC + | Caucasian | PROVE 3 | SVR | 29 |
| | Telaprevir | | | positive | |
| E1 | SOC + | African | PROVE 1 & 2 | SVR | 11 |
| | Telaprevir | American | | negative | |
| E2 | SOC + | African | PROVE 3 | SVR | 8 |
| | Telaprevir | American | | negative | |
| E3 | SOC + | African | PROVE 1 & 2 | SVR | 8 |
| | Telaprevir | American | | positive | |
| E4 | SOC + | African | PROVE 3 | SVR | 8 |
| | Telaprevir | American | | positive | |

### Cellcarta Analysis

Sample Preparation and Mass Spectrometry

All samples were depleted of abundant proteins with two sequential antibody columns (IgY14 and Supermix, Sigma), and the remaining lower abundance proteins were digested with trypsin. Each sample was then further fractionated by reversed phase liquid chromatography, coupled by electrospray to a Waters QTOF mass spectrometer (LC-MS).

Data Analysis

Chromatographic component ions were detected and matched across all samples and compared for relative peak intensity. Peak intensity was normalized to account for small differences in protein concentration between samples. A multifactor ANOVA analysis was then applied to identify components that were differentially expressed between the groups of interest. High stringency thresholds were used to ensure the statistical significance of the identified components. Details of these steps are provided below.

Seven samples were omitted from the statistical analysis. These samples appeared to contain very high abundance proteins that were not completely removed by the immunoaffinity depletion. In the LC-MS, peptides from these proteins suppressed the signal of the remaining peptides. Leaving these samples in the statistical analysis would have reduced the quality of the results and were therefore removed.

Normalization

All intensity values are log (base e) transformed with values<0 replaced by 0. The sum of the intensities for each sample is then calculated. In this study, samples lying between the 25th and 75th percentiles were used to create an average sample (i.e. the Reference sample), against which the remaining real samples were then normalized. The normalization factors are chosen in such a way that the log ratios between the real and the Reference sample over all the components is adjusted to 0.

Statistical Analysis

A T-test was used to determine the differentially expressed components for various comparisons defined in the study objectives. The FDR and q-values are calculated based on the p-values obtained from the T-test, using Storey's method to make multiple testing adjustments (implemented in MATLAB).

Thresholds/Cutoffs

Thresholds used to determine which components were differentially expressed within the various comparisons consisted of a differential expression of at least 1.8 (ANOVA), a maximum q-value of 0.1 (Storey's method), a component intensity 70 in at least 5 samples and a charge 2. For three comparisons, (D1 vs B), (D2 vs B) and (D1,D2 vs B), selection thresholds were relaxed to include q-values up to 0.4. This was due to the low number of significant differences observed with the more strict criteria.

Sequencing and Protein Identification

In addition to the peptides found to be differentially expressed by the statistical analyses described above, a list of additional components were selected using Lasso Regression Classifier Algorithms (“LRCA”) to try to identify the peptides that best predicted response to therapy. All or nearly all of the LRCA-selected peptides overlap with the differentially expressed peptides identified by statistical analysis. This list of additional components also was included in the peptide sequencing. The differentially expressed components were targeted for sequencing on a Waters QTOF mass spectrometer and/or Orbitrap XL (Thermo) mass spectrometer and the resulting fragmentation patterns were matched to the corresponding peptide sequences found in a database composed of the IPI (International Protein Index) human proteins (version 3.68) and the NCBI HCV genotype 1 database (taxonomy ID 41865).

### Results and Discussion

A total of 8374 chromatographic components were detected, matched across all samples and compared for relative peak intensity. Normalized intensity data for each component was previously delivered. Statistical comparisons performed to detect components relevant to the objectives are summarized in Table 2. A total of 1350 of these components were found to meet one or more criteria for differential expression, using the selected cutoffs.

TABLE 2
Number of differentially expressed components addressing the study objectives.
Patient groups indicated in the Comparison column refer to those described in Table
1. Numbers of differentially expressed components found between parentheses were
obtained with a more relaxed q-value < 0.4. FC, fold change; q, q-value.

| | | | Number of differently | |
|---|---|---|---|---|
| | | | expressed components | |
| | | | FC > 2 | FC > 1.8, |
| Objective description | | Comparison | q < 0.05 | q < 0.1 |
| 1. | Identify predictive biomarkers of | D1 vs C1 | 10 | 32 |
| | SVR or non-response in patients | D2 vs C2 | 20 | 71 |
| | treated with SOC plus Telaprevir | (D1, D2) vs | 267 | 558 |
| | | (C1, C2) | | |
| | | (D1, D2, E3, E4) vs | 25 | 109 |
| | | (C1, C2, E1, E2) | | |
| 2. | Identify predictive biomarkers | B vs A | 197 | 423 |
| | of SVR or non-response in | | | |
| | patients treated with SOC | | | |
| 3. | Distinguish between SVR positive | (D1, D2) vs B | 0 | 5 (188) |
| | patients receiving SOC alone as | D1 vs B | 0 | 0 (0) |
| | compared with SOC plus Telaprevir | D2 vs B | 322 | 561 (675) |
| 4. | Identify predictive biomarkers | (B1, D1, D2, E3, E4) vs | 102 | 219 |
| | of response to treatment | (A, C1, C2, E1, E2) | | |
| | | (B, D1, D2) vs | 227 | 419 |
| | | (A, C1, C2) | | |

Peptide sequences were obtained for a total of 239 components. Thirteen peptides were from immunoglobulins and were removed. Upon clustering, the remaining 226 peptides were found to represent 71 proteins, with three (CFI, SERPINA6, FCGR2B, see Table 5) being uniquely identified by peptides from the Lasso Regression Classifier Algorithms analysis. The remaining list of 68 proteins is shown in Table 3, including two that are not differentially expressed at the protein level but contain differentially expressed peptides (C1RL and SERPINA7). Differential expression at the protein level is based on a categorical cut-off of 1.5 fold difference (up or down) as represented by the median differential expression for all peptides associated with (putatively originating from) that protein by alignment of the amino acid sequences of the peptide and protein. These proteins are discussed below in separate sections corresponding to the objectives of this study. Because of the similar nature of objectives 1 and 4, both pertaining to biomarkers of response to treatment, these objectives are discussed together.

Overall, the majority of proteins identified in this study are known to be found in plasma. Importantly, nearly all are annotated as originating from the liver, consistent with liver damage by the HCV infection (Table 4).

Objectives 1 and 4: Identify Predictive Biomarkers of Response to Treatment

A total of 34 proteins distinguish Caucasian Telaprevir-treated patients who achieved SVR from those who did not (Table 3, first column, D1,D2 vs C1,C2).

| Gene Symbol | Gene ID |
|---|---|
| LPA | 4018 |
| CNDP1 | 84735 |
| TPM4 | 7171 |
| GAPDH | 2597 |
| FKBP1A | 2280 |
| PARVB | 29780 |
| VCP | 7415 |
| PPIA | 5478 |
| PFN1 | 5216 |
| CAP1 | 10487 |
| ILK | 3611 |
| PLEK | 5341 |
| GSTP1 | 2950 |
| TLN1 | 7094 |
| ZYX | 7791 |
| CLIC1 | 1192 |
| F13A1 | 2162 |
| VCL | 7414 |
| FLNA | 2316 |
| SDPR | 8436 |
| TAGLN2 | 8407 |
| C9 | 735 |
| CP | 1356 |
| YWHAE | 7531 |
| ORM1 | 5004 |
| HPR | 3250 |
| FERMT3 | 83706 |
| A2M | 2 |
| SERPINA1 | 5265 |
| LGALS3BP | 3959 |
| CTSD | 1509 |
| FTL | 2512 |
| CHI3L1 | 1116 |
| FCGBP | 8857 |

Only 6 of these proteins remain significant, however, in a comparison of either the PROVE 1&2 patients (20 vs 9 patients) (D1 vs C1 in Table 3) or the PROVE 3 patients alone (29 vs 29 patients) (D2 vs. C2 in Table 3), suggesting that the power of the study is greatly enhanced with 49 vs 38 patients in the comparison.

A first glance at Table 3 suggests that many of these proteins also appear to distinguish SVR+/− patients who received either SOC or Telaprevir/SOC (B,D1,D2 vs A,C1,C2). Likewise, addition of the African American cohorts to the Caucasian comparison also seems to retain some of the markers of the Caucasian group alone. However, B vs A alone (SOC treated SVR+/−) has little overlap with the Telaprevir treated patient comparison. One possible interpretation is that addition of the SOC-alone or African American groups is diluting the effect of the Telaprevir-treated Caucasian comparison. Adding the SOC groups reduces the number of significant proteins that separate SVR+/− from 34 to 27. (Table 3, second biomarker analysis column.) Addition of the African American population to the Caucasian group reduces the number of significant proteins to 13. (Table 3, fourth biomarker analysis column.) Thus, many of the treatment response markers appear to be specifically related to predicting SVR in the Telaprevir-treated Caucasian population.

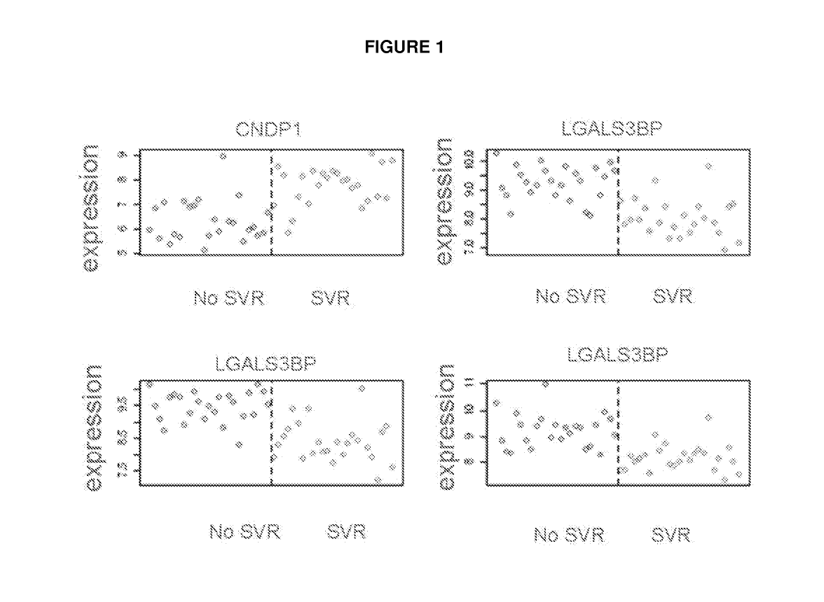

Along with the predictive markers are multiple prognostic markers of treatment response, independent of treatment (Telaprevir/SOC or SOC alone). The two strongest such prognostic markers are CNDP1 and LGALS3BP. FIG. 1 depicts the intensities for HCV-infected subjects of the 4 peptides identified in Example 1 that were the best predictors of response to the standard of care (SOC) HCV treatment. Below is a table showing how well these 4-peptides work in a linear model built using the lasso regression algorithm to predict outcome to standard-of-care therapy.

| | | misclassification rate | | |
|---|---|---|---|---|
| Lambda | # | | training w/cross | test set |
| (adj. parameter) | peptides | training | validation (std dev) | (prove 3) |
| 0.026 | 4 | 0.14 | 0.20 (0.06) | 0.24 |

TABLE 3
Complete list of proteins identified. Numbers indicate the differential intensity ratios (dI) obtained in the
specified comparisons. Statistically significant dI values are lightly shaded and italicized if upregulated; or darkly shaded and bold if
downregulated by at least 1.5 fold.

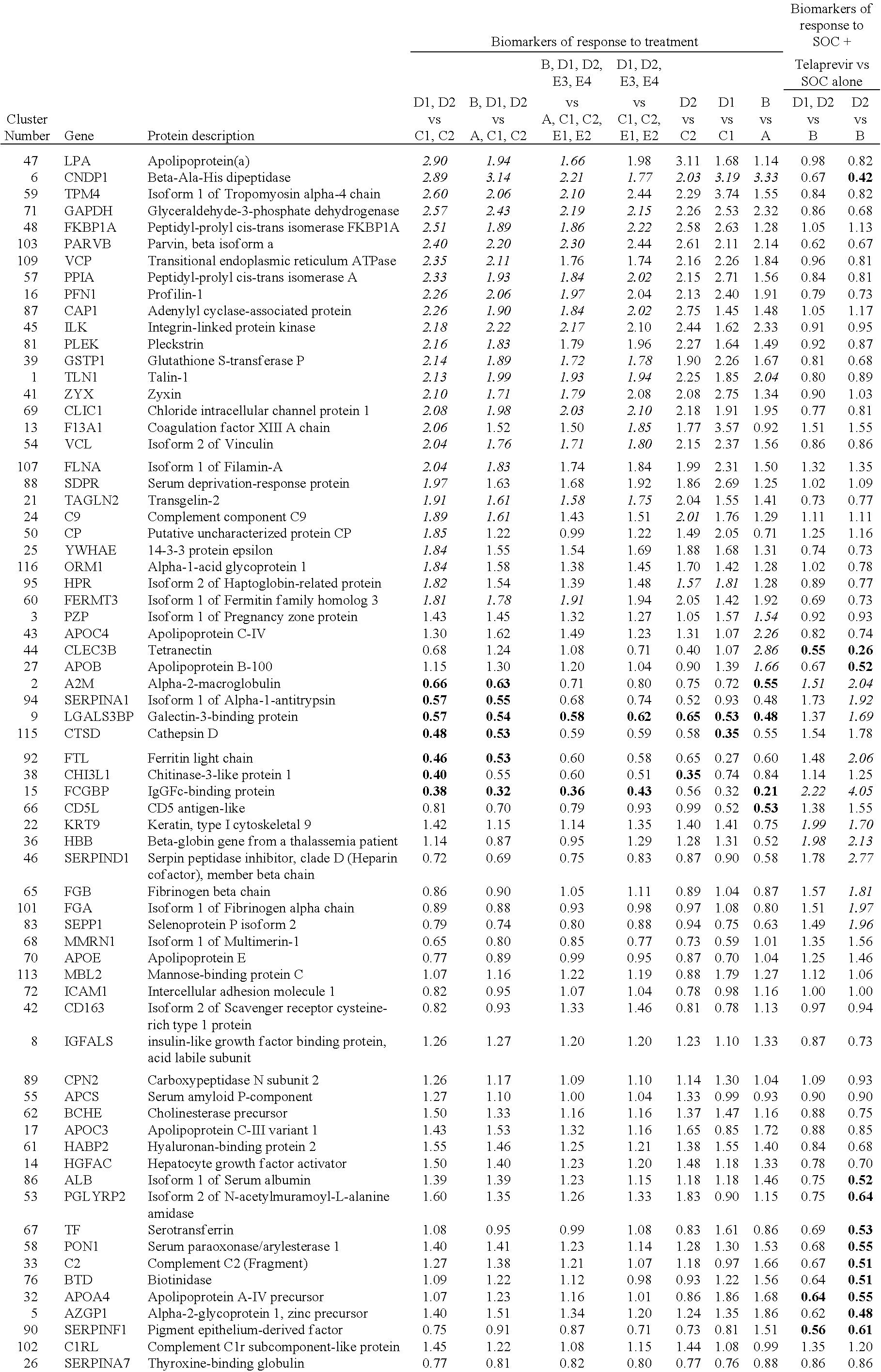

TABLE 4
Association of candidate biomarkers with liver origin,
plasma localization, and various cellular functions.

| | | | | | | Complement | | | | |
|---|---|---|---|---|---|---|---|---|---|---|
| | | | | | Response | and | | Scavenger | Iron | Acute |
| Cluster | | | | | to | coagulation | Lipid | receptor | homeo- | inflammatory |
| ID | Gene Name | Protein description | Liver | Plasma | wounding | cascade | binding | activity | stasis | response |
| 2 | A2M | Alpha-2-macroglobulin | X | X | X | X | | | | X |
| 86 | ALB | Isoform 1 of Serum albumin | X | | | | X | | | |
| 55 | APCS | Serum amyloid P-component | X | X | X | | | | | X |
| 32 | APOA4 | Apolipoprotein A-IV precursor | X | | | | X | | | |
| 27 | APOB | Apolipoprotein B-100 | X | X | | | X | | | |
| 17 | APOC3 | Apolipoprotein C-III | X | | | | X | | | |
| 43 | APOC4 | Apolipoprotein C-IV | X | X | | | X | | | |
| 70 | APOE | Apolipoprotein E | X | | | | X | | X | |
| 5 | AZGP1 | alpha-2-glycoprotein 1 zinc | X | X | | | X | | | |
| | | precursor | | | | | | | | |
| 62 | BCHE | Cholinesterase precursor | X | X | | | | | | |
| 76 | BTD | Biotinidase | X | X | | | | | | |
| 102 | C1RL | Complement C1r subcomponent- | X | X | X | | | | | X |
| | | like protein | | | | | | | | |
| 33 | C2 | Complement C2 (Fragment) | X | X | X | X | | | | X |
| 24 | C9 | Complement component C9 | X | X | X | X | | | | X |
| 87 | CAP1 | Adenylyl cyclase-associated | X | | | | | | | |
| | | protein | | | | | | | | |
| 42 | CD163 | Isoform 2 of Scavenger receptor | X | X | X | | | X | | X |
| | | cysteine-rich type 1 protein M130 | | | | | | | | |
| 66 | CD5L | CD5 antigen-like | X | | | | | X | | |
| 38 | CHI3L1 | Chitinase-3-like protein 1 | | | | | | | | |
| 44 | CLEC3B | Tetranectin | | X | | | | | | |
| 69 | CLIC1 | Chloride intracellular channel | X | | | | | | | |
| | | protein 1 | | | | | | | | |
| 6 | CNDP1 | Beta-Ala-His dipeptidase | | X | | | | | | |
| 50 | CP | Highly similar to | X | X | | | | | X | |
| | | CERULOPLASMIN | | | | | | | | |
| 89 | CPN2 | Carboxypeptidase N subunit 2 | X | X | | | | | | |
| 115 | CTSD | Cathepsin D | X | X | | | | | | |
| 13 | F13A1 | Coagulation factor XIII A chain | | X | X | X | | | | |
| 15 | FCGBP | IgGFc-binding protein | | X | | | | | | |
| 60 | FERMT3 | Isoform 1 of Fermitin family | | | | | | | | |
| | | homolog 3 | | | | | | | | |
| 101 | FGA | Isoform 1 of Fibrinogen alpha | X | X | X | X | | | | |
| | | chain | | | | | | | | |
| 65 | FGB | Fibrinogen beta chain | X | X | X | X | | | | |
| 48 | FKBP1A | Peptidyl-prolyl cis-trans isomerase | | | | | | | X | |
| | | FKBP1A | | | | | | | | |
| 107 | FLNA | Filamin A, alpha | X | | | | | | | |
| 92 | FTL | Ferritin light chain | X | | | | | | X | |
| 71 | GAPDH | Glyceraldehyde-3-phosphate | X | | | | | | | |
| | | dehydrogenase | | | | | | | | |
| 39 | GSTP1 | Glutathione S-transferase P | X | | | | | | | |
| 61 | HABP2 | Hyaluronan-binding protein 2 | | X | | | | | | |
| 36 | HBB | Hemoglobin subunit beta | | | | | | | | |
| 14 | HGFAC | Hepatocyte growth factor activator | X | X | | | | | | |
| 95 | HPR | Isoform 2 of Haptoglobin-related | X | X | | | | | X | |
| | | protein | | | | | | | | |
| 72 | ICAM1 | Intercellular adhesion molecule 1 | X | X | | | | | | |
| 8 | IGFALS | insulin-like growth factor binding | X | X | | | | | | |
| | | protein, acid labile subunit isoform | | | | | | | | |
| | | 1 precursor | | | | | | | | |
| 45 | ILK | Integrin-linked protein kinase | | | | | | | X | |
| 22 | KRT9 | Keratin, type I cytoskeletal 9 | | | | | | | | |
| 9 | LGALS3BP | Galectin-3-binding protein | X | X | | | | X | | |
| 47 | LPA | Apolipoprotein(a) | X | | X | | | | | |
| 113 | MBL2 | Mannose-binding protein C | X | X | X | X | | | | X |
| 68 | MMRN1 | Isoform 1 of Multimerin-1 | X | X | X | | | | | |
| 116 | ORM1 | Orosomucoid 1 precursor | X | X | X | | | | | X |
| 103 | PARVB | Beta-parvin | X | | | | | | | |
| 16 | PFN1 | Profilin-1 | X | | | | | | | |
| 53 | PGLYRP2 | Isoform 2 of N-acetylmuramoyl-L- | X | X | | | | | | |
| | | alanine amidase | | | | | | | | |
| 81 | PLEK | Pleckstrin | | | X | | X | | | |
| 58 | PON1 | Serum paraoxonase/arylesterase 1 | X | X | | | X | | | |
| 57 | PPIA | Peptidyl-prolyl cis-trans | X | | | | | | | |
| | | isomerase A | | | | | | | | |
| 3 | PZP | Isoform 1 of Pregnancy zone | X | X | | | | | | |
| | | protein | | | | | | | | |
| 88 | SDPR | Serum deprivation-response protein | | | | | X | | | |
| 83 | SEPP1 | Selenoprotein P | X | X | | | | | | |
| 94 | SERPINA1 | Isoform 1 of Alpha-1-antitrypsin | X | X | X | X | | | | X |
| 26 | SERPINA7 | Thyroxine-binding globulin | X | X | | | | | | |
| 46 | SERPIND1 | Serpin peptidase inhibitor, clade D | X | X | X | X | | | | |
| | | (Heparin cofactor), member 1 | | | | | | | | |
| 90 | SERPINF1 | Pigment epithelium-derived factor | X | X | | | | | | |
| 21 | TAGLN2 | Transgelin-2 | X | | | | | | | |
| 67 | TF | Serotransferrin | X | X | X | | | | X | X |
| 1 | TLN1 | Talin-1 | X | | | | | | | |
| 59 | TPM4 | Isoform 1 of Tropomyosin alpha-4 | | | | | | | | |
| | | chain | | | | | | | | |
| 54 | VCL | Isoform 2 of Vinculin | X | | | | | | | |
| 109 | VCP | Transitional endoplasmic reticulum | X | | | | X | | | |
| | | ATPase | | | | | | | | |
| 25 | YWHAE | 14-3-3 protein epsilon | X | | | | | | | |
| 41 | ZYX | Zyxin | | | | | | | | |

Objective 2: Identify Predictive Response Markers in Patients Treated with SOC

Comparison of the plasma proteomes of responder and non-responder Caucasians receiving SOC resulted in the identification of a small number of differentially expressed proteins (Table 3, B vs A comparison). Four proteins were decreased in the responder group (Group B in Table 3), and 6 proteins were increased in the same group.

| Gene Symbol | Gene ID |
|---|---|
| CNDP1 | 84735 |
| TLN1 | 7094 |
| PZP | 5858 |
| APOC4 | 346 |
| CLEC3B | 7123 |
| APOB | 338 |
| A2M | 2 |
| LGALS3BP | 3959 |
| FCGBP | 8857 |
| CD5L | 922 |

Of the four proteins that decreased in the responders, three of them (A2M, CD5L, LGALS3BP) have been previously associated with liver physiology or dysfunction. Increased serum A2M (α-2-macroglobulin), for example, has been shown to be associated with liver fibrosis (Gangadharan et al., 2007a; Rossi et al., 2003). Increased A2M has also been recently associated with response to INF-β treatment (Gandhi et al., 2010), suggesting that treatments can affect this protein's expression levels. CD5L is believed to have an immune regulatory role, and increased serum levels of this protein have been associated with HCV-induced liver damage (Gangadharan et al., 2007; Cheung et al., 2009). This protein was also down in patients that went on to respond to Telaprevir, but the difference was not as statistically significant. LGALS3BP (Galectin-3-binding protein) is a secreted protein that forms part of the extracellular matrix, and may be involved in stimulating the host defense against viruses and tumor cells. Serum levels of Galectin-3-binding protein are known to become elevated in patients with hepatic carcinomas, cirrhosis, or HCV infections (Iacovazzi et al., 2001; Kittl et al., 2000.) The fourth protein to show a decrease in the plasma of the responder patients in this comparison, FCGBP (IgGFc-binding protein), does not have any known links to liver dysfunction or response to treatment. This protein appears to be primarily expressed in the colon epithelia, as a component of the two mucus layers generated by that tissue (Johansson et al., 2009). However, serum levels of FCGBP have been shown to increase in patients with rheumatoid arthritis and systemic lupus erythematosus (Kobayashi et al., 2001). Taken together, therefore, decreased levels of four proteins associated with tissue damage may suggest that response to treatment is more likely when initial levels of tissue damage are low.

Of the 6 proteins found to be increased in the sera of the responder patients in this comparison, three appear to follow the same pattern as those above to various levels. CNDP1, a hepatic peptidase, has been reported to decrease in patients with cirrhosis and hepatoma (Bando et al., 1986). Plasma levels of CLEC3B (tetranectin), a secretion carrier protein, appear to vary more widely in patients with cirrhosis than in normal controls, although overall this protein did not show a significant population based reduction (Kluft et al., 1989). PZP (pregnancy zone protein) is a member of the α-2-macroglobulin family, and may therefore be involved in liver pathology in a similar manner, although no specific information relating to tissue damage was found in our analysis. As for the other three proteins, all appear to be related in various ways with viral infection. APOC4 (apolipoprotein C-IV) has reports indicating its liver mRNA transcript increased during HCV infection (Kim et al., 2008). APOB has been described as essential for the formation of infectious HCV particles (Popescu and Dubuisson, 2009), although it is not known how that may affect the serum levels of this protein during disease progression and treatment. TLN1 (talin-1), a cytoskeletal protein, has been found to be elevated in PBMCs with HIV infection (Zhang et al., 2010).

Objective 3: Identify Differences Between Responder Caucasians Receiving SOC or SOC Plus Telaprevir

For this objective, responder patients that received SOC+Telaprevir in either the Prove 1 & 2 trials or the Prove 3 trial were compared to responder patients from the Prove 1 & 2 trial that received only SOC. No statistically significant proteins were identified when comparing patients within the same trial (1&2 or 3). Only when the comparison involves a comparison across studies (1&2 vs 3) do significant differences appear. Thus, the proteins identified herein as associated with these differences would benefit from further validation. Rather than being related to treatment, the differences could be related to differences in sample collection or handling between the two trials. While the list of differentially expressed proteins contains mainly proteins associated with liver function or the resolution of a tissue injury, including the associated inflammatory/immune response, it also includes beta globin and fibrinogen alpha and beta. These last three proteins could point to differences in sample handling and the preparation of plasma from the collected blood in the two sets of trials.

In addition to the peptides found to be differentially expressed as described above, a list of 62 chromatographic components were sequenced. Sequencing of these components was carried out first on a Waters QTOF mass spectrometer and then on a Thermo Orbitrap XL mass spectrometer. Forty-two components were assigned a peptide sequence and contributed to the identification of 11 proteins (Table 5). Eight of these proteins include peptides on the list of targeted components from the trypsin-digest study. Three proteins are unique to the Lasso Regression Classifier Algorithm list of peptides, but are represented by only one sequenced peptide each (CFI, SERPINA6, FCGR2B). By way of comparison, for example, 19 masses that were statistically significant were identified as corresponding to LGALS3BP, providing a very strong indication that the masses truly were derived from this protein and that the differential expression data is truly representative of that protein.

TABLE 5
Peptides of the LRCA-developed list which were matched
to a protein sequence.
* indicates a protein identified uniquely by peptides of the LRCA list.

| Component ID | Peptide sequence | Protein | SEQ ID NO |
|---|---|---|---|
| 7932784 | QVEGM[147.0354]EDWKQDSQLQK | AZGP1 | 1 |
| 7920687 | VFSLQWGEVK | CFI* | 2 |
| 7934555 | FPLTNAIK | CHI3L1 | 3 |
| 7921305 | ALEQDLPVNIK | CNDP1 | 4 |
| 7929455 | FLEMAQLH | CNDP1 | 5 |
| 7934183 | GDGWLTDPYVLTEVDGK | CNDP1 | 6 |
| 7921919 | MMAVAADTLQR | CNDP1 | 7 |
| 7922664 | SVVLIPLGAVDDGEHSQNEK | CNDP1 | 8 |
| 7945659 | SVVLIPLGAVDDGEHSQNEK | CNDP1 | 9 |
| 7923790 | TVFGTEPDMIR | CNDP1 | 10 |
| 7920901 | VFQYIDLHQDEFVQTLK | CNDP1 | 11 |
| 7930579 | VFQYIDLHQDEFVQTLK | CNDP1 | 12 |
| 7922644 | WNYIEGTK | CNDP1 | 13 |
| 7922733 | YPSLSIHGIEGAFDEPGTK | CNDP1 | 14 |
| 7923300 | AVPPNNSNAAEDDLPTVELQGVVPR | F13A1 | 15 |
| 7934516 | CSVQNGLLGCYPDR | FCGBP | 16 |
| 7924448 | VTFFQN[115.0269]GK | FCGR2B* | 17 |
| 7919798 | ASHEEVEGLVEK | LGALS3BP | 18 |
| 7919989 | ELSEALGQIFDSQR | LGALS3BP | 19 |
| 7922782 | ELSEALGQIFDSQR | LGALS3BP | 20 |
| 7931054 | ELSEALGQIFDSQR - H2O | LGALS3BP | 21 |
| 7919766 | IDITLSSVK | LGALS3BP | 22 |
| 7923083 | KTLQALEFHTVPFQLLAR | LGALS3BP | 23 |
| 7923401 | LAS AYGAR | LGALS3BP | 24 |
| 7920007 | SQLVYQSR | LGALS3BP | 25 |
| 7919825 | STHTLDLSR | LGALS3BP | 26 |
| 7922000 | STSSFPCPAGHFN[115.0269]GFR | LGALS3BP | 27 |
| 7924468 | STSSFPCPAGHFN[115.0269]GFR | LGALS3BP | 28 |
| 7923827 | STSSFPCPAGHFNGFR | LGALS3BP | 29 |
| 7919767 | TLQALEFHTVPF | LGALS3BP | 30 |
| 7922931 | TLQALEFHTVPFQLLAR | LGALS3BP | 31 |
| 7922736 | VADVTDFEGWK | LGALS3BP | 32 |
| 7919640 | VEIFYR | LGALS3BP | 33 |
| 7920263 | YYPYQSFQTPQHPSFLFQDK | LGALS3BP | 34 |
| 7927752 | YYPYQSFQTPQHPSFLFQDK | LGALS3BP | 35 |
| 7920209 | YYPYQSFQTPQHPSFLFQDKR | LGALS3BP | 36 |
| 7921049 | YYPYQSFQTPQHPSFLFQDKR | LGALS3BP | 37 |
| 7925800 | AVGYLITGYQR | PZP | 38 |
| 7944971 | M[147.0354]DPNAAYVNM | SERPINA6* | 39 |
| | [147.0354]SNHHR | | |
| 7921901 | EGQM[147.0354]ESVEAAM | SERPINA7 | 40 |
| | [147.0354]SSK | | |
| 7921930 | EGQMESVEAAM[147.0354]SSK | SERPINA7 | 41 |
| 7921639 | MGIQHAYSENADFSGLTEDN[115.0269] | SERPINA7 | 42 |
| | GLKLSNAAHK | | |

### Conclusions

The predominant set of candidate biomarkers identified in this study is related to predicting SVR in Caucasian patients treated with Telaprevir. A smaller number were found that can distinguish SVR in SOC-treated patients and half of these are unique to that comparison. In addition to treatment-specific predictive markers, more general prognostic markers for SVR were found.

### REFERENCES CITED IN EXAMPLE 1

Aliya S, Reddanna P, Thyagaraju K. Does glutathione S-transferase Pi (GST-Pi) a marker protein for cancer? Mol Cell Biochem. 2003, 253(1-2):319-27.
Bando K, Ichihara K, Toyoshima H, Shimotuji T, Koda K, Hayashi C, Miyai K. Decreased activity of carnosinase in serum of patients with chronic liver disorders. Clin Chem. 1986, 32(8):1563-5.
Cheung K J, Tilleman K, Deforce D, Colle I, Van Vlierberghe H. The HCV serum proteome: a search for fibrosis protein markers. J Viral Hepat. 2009, 16(6):418-29.
Domené H M, Hwa V, Argente J, Wit J M, Camacho-Hübner C, Jasper H G, Pozo J, van Duyvenvoorde H A, Yakar S, Fofanova-Gambetti O V, Rosenfeld R G; International ALS Collaborative Group. Human acid-labile subunit deficiency: clinical, endocrine and metabolic consequences. Horm Res. 2009, 72(3):129-41.
Gangadharan B, Antrobus R, Dwek R A, Zitzmann N. Novel serum biomarker candidates for liver fibrosis in hepatitis C patients. Clin Chem. 2007, 53(10):1792-9.
Gandhi K S, McKay F C, Diefenbach E, Crossett B, Schibeci S D, Heard R N, Stewart G J, Booth D R, Arthur J W. Novel approaches to detect serum biomarkers for clinical response to interferon-beta treatment in multiple sclerosis. PLoS One. 2010, 5(5):e10484.
Iacovazzi P A, Trisolini A, Barletta D, Elba S, Manghisi O G, Correale M. Serum 90K/MAC-2BP glycoprotein in patients with liver cirrhosis and hepatocellular carcinoma: a comparison with alpha-fetoprotein. Clin Chem Lab Med. 2001, 39(10):961-5.
Johansson M E, Thomsson K A, Hansson G C. Proteomic analyses of the two mucus layers of the colon barrier reveal that their main component, the Muc2 mucin, is strongly bound to the Fcgbp protein. J Proteome Res. 2009, 8(7):3549-57.
Kataoka H, Kawaguchi M. Hepatocyte growth factor activator (HGFA): pathophysiological functions in vivo. FEBS J. 2010, 277(10):2230-7.
Kawaguchi T, Yamagishi S, Sata M. Branched-chain amino acids and pigment epithelium-derived factor: novel therapeutic agents for hepatitis c virus-associated insulin resistance. Curr Med Chem. 2009, 16(36):4843-57.
Keil C, Maskos K, Than M, Hoopes J T, Huber R, Tan F, Deddish P A, Erdös E G, Skidgel R A, Bode W. Crystal structure of the human carboxypeptidase N (kininase I) catalytic domain. J Mol Biol. 2007, 366(2):504-16.
Kim E, Li K, Lieu C, Tong S, Kawai S, Fukutomi T, Zhou Y, Wands J, Li J. Expression of apolipoprotein C-IV is regulated by Ku antigen/peroxisome proliferator-activated receptor gamma complex and correlates with liver steatosis. J Hepatol. 2008, 49(5):787-98.
Kittl E M, Hofmann J, Hartmann G, Sebesta C, Beer F, Bauer K, Huber K R. Serum protein 90K/Mac-2BP is an independent predictor of disease severity during hepatitis C virus infection. Clin Chem Lab Med. 2000, 38(3):205-8.
Kluft C, Los P, Clemmensen I, Brommer E J, Gevers Leuven J A, Boks A L, Vellenga E. Quantitation of plasma levels of tetranectin—effects of oral contraceptives, pregnancy, treatment with L-asparaginase and liver cirrhosis. Thromb Haemost. 1989, 62(2):792-6.
Kobayashi K, Yagasaki M, Harada N, Chichibu K, Hibi T, Yoshida T, Brown W R, Morikawa M. Detection of Fcgamma binding protein antigen in human sera and its relation with autoimmune diseases. Immunol Lett. 2001, 79(3):229-35.
Popescu C I, Dubuisson J. Role of lipid metabolism in hepatitis C virus assembly and entry. Biol Cell. 2009, 102(1):63-74.
Rossi E, Adams L, Prins A, Bulsara M, de Boer B, Garas G, MacQuillan G, Speers D, Jeffrey G. Validation of the FibroTest biochemical markers score in assessing liver fibrosis in hepatitis C patients. Clin Chem. 2003, 49(3):450-4.
Sakai M, Muramatsu M. Regulation of glutathione transferase P: a tumor marker of hepatocarcinogenesis. Biochem Biophys Res Commun. 2007, 357(3):575-8.
Sasaki M, Ikeda H, Kataoka H, Nakanuma Y. Augmented expression of hepatocytes growth factor activator inhibitor type 1 (HAI-1) in intrahepatic small bile ducts in primary biliary cirrhosis. Virchows Arch. 2006, 449(4):462-71.
Zhang L, Jia X, Zhang X, Sun J, Peng X, Qi T, Ma F, Yin L, Yao Y, Qiu C, Lu H. Proteomic analysis of PBMCs: characterization of potential HIV-associated proteins. Proteome Sci. 2010, 8:12-23.

### EXAMPLE 2

The experiments described in Example 1 are repeated with the following modification. Instead of measuring peptide components from protease-digested plasma, the experimental protocol involves measuring the concentration of the proteins identified in Example 1 as predictive biomarkers. In some variations, proteins are measured in blood, serum, or plasma using an immunoassay for the protein, such as an ELISA assay. For enzymatic proteins, protein may alternatively be measured indirectly with an enzymatic assay.

In still another variation, the experiment is repeated with other biological samples. For example, the experiments are repeated to measure the biomarkers in tissue biopsy, e.g., from the liver; in urine; or in feces.

Biomarkers that are predictive of successful HCV therapy are confirmed, and data is collected reflecting measurements of the markers in responders and non-responders to continually improve the predictive model.

### EXAMPLE 3

The experiments described in Examples 1 and 2 are repeated for other treatment regimen(s) to identify protein markers that are useful for predicting responders to such other treatment regimens. For example, the experiments are repeated Telaprevir alone; Telaprevir plus an interferon; or Telaprevir plus Ribavirin.

1. A method of evaluating a human subject infected with hepatitis C virus (HCV) for a treatment, the method comprising:
(a) measuring at least one biomarker from at least one biological sample isolated from a human subject who is infected with HCV, wherein the at least one biomarker is selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof; and
(b) determining an elevated probability of achieving sustained viral response (SVR) from treatment for HCV infection from the output of a model, wherein the inputs to said model comprise said measurement(s) of the at least one biomarker.

2. A method of evaluating a human subject infected with hepatitis C virus (HCV) for a treatment, the method comprising:
(a) measuring at least one biomarker from at least one biological sample isolated from a human subject who is infected with HCV, wherein the at least one biomarker is selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof; and
(b) determining an elevated probability of achieving sustained viral response (SVR) from treatment for HCV infection from the output of a model, wherein the inputs to said model comprise said measurement(s) of the at least one biomarker.

3.-8. (canceled)

9. The method according to claim 2, wherein the at least one biomarker is selected from the group consisting of A2M, CD5L, LGALS3BP, CNDP1, and CLEC3B.

10.-12. (canceled)

13. The method according to claim 1, wherein the method further comprises measuring at least one supplemental biomarker selected from the group consisting of apolipoprotein A1, haptoglobin, total bilirubin, and γ-glutamyl-transpeptidase (GGT) in the biological sample, wherein the inputs of the model further include the measurement(s) of the at least one supplemental biomarker, and wherein the study of the population of human subjects included data collection about the at least one supplemental biomarker.

14. The method according to claim 1, wherein the method further comprises obtaining a measurement of at least one clinical parameter of the subject selected from the group consisting of: sex, age, race, weight, body mass index, height, weight, hip circumference, waist circumference, history of tobacco usage, history of alcohol consumption, exercise pattern, presence of diabetes, fasting glucose, triglycerides, fibrosis score, and HCV viral load, wherein inputs of the model further include the measurement(s) of the at least one clinical parameter, and wherein the study of the population of human subjects included data collection about the at least one clinical parameter.

15. (canceled)

16. The method according to claim 1, wherein the method further comprises obtaining a measurement of alanine transaminase (ALT), Aspartate Aminotransferase (AST), and combinations thereof from the at least one biological sample, wherein the inputs of the model further include the measurement of ALT and/or AST, and wherein the study of the population of human subjects included data collection about ALT and/or AST.

17. The method according to claim 1, wherein the method further comprises obtaining a measurement of Carbohydrate-deficient transferrin (CDT) from the at least one biological sample, wherein the inputs of the model further include the measurement of CDT, and wherein the study of the population of human subjects included data collection about CDT.

18. (canceled)

19. The method according to claim 1, further comprising a step, prior to the measuring the biomarkers, of obtaining at least one biological sample from the subject.

20. The method according to claim 1, wherein the biological sample comprises whole blood or a blood component selected from serum and plasma.

21. (canceled)

22. The method according to claim 1, wherein at least one of said biomarker measurements is obtained by an immunoassay.

23. The method according to claim 22, wherein at least one of said biomarkers is an enzyme, and wherein the enzyme is measured by an enzymatic activity assay.

24.-28. (canceled)

29. The method according to claim 1, wherein the elevated probability is at least 65% probability of sustained viral response (SVR) six months after cessation of the therapy.

30. A method of treating HCV infection in a human subject comprising:
(a) measuring at least one marker in a biological sample from a human subject infected with HCV, wherein the at least one marker is selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof;
(b) determining an elevated probability of achieving sustained viral response (SVR) from treatment for HCV infection; and
(c) administering a composition comprising telaprevir to the subject if the measurement(s) of the at least one biomarker indicates a probability of at least 65% of sustained viral response (SVR) six months after cessation of the therapy.

31. A method of treating HCV infection in a human subject comprising:
(a) measuring at least one marker in a biological sample from a human subject infected with HCV, wherein the at least one marker is selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof;
(b) determining an elevated probability of achieving sustained viral response (SVR) from treatment for HCV infection and
(c) administering a treatment comprising an interferon and ribavirin to the subject if the measurement(s) of the at least one biomarker indicates a probability of at least 65% of sustained viral response (SVR) six months after cessation of the therapy.

32. A kit comprising reagents for measuring at least 2, at least 3, at least 4, at least 5, at least 6, at least 7, at least 8, at least 9, or at least ten biomarkers packaged together, wherein the biomarkers are selected from the group consisting of: LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof.

33. A kit comprising reagents for measuring at least 2, at least 3, at least 4, at least 5, at least 6, at least 7, at least 8, at least 9, or at least ten biomarkers packaged together, wherein the biomarkers are selected from the group consisting of: carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof.

34.-37. (canceled)

38. A medical diagnostic test system for evaluating likelihood of benefit of a therapy for hepatitis C in a human subject infected with hepatitis C virus (HCV), the system comprising:
a data collection tool adapted to collect biomarker and clinical measurement data representative of measurements of biomarkers and clinical parameters from a human subject, wherein said biomarkers comprise at least one marker selected from the group consisting of: wherein the at least one biomarker is selected from the group consisting of LPA, CNDP1, TPM4, GAPDH, FKBP1A, PARVB, VCP, PPIA, PFN1, CAP1, ILK, PLEK, GSTP1, TLN1, ZYX, CLIC1, F13A1, VCL, FLNA, SDPR, TAGLN2, C9, CP, YWHAE, ORM1, HPR, FERMT3, A2M, SERPINA1, LGALS3BP, CTSD, FTL, CHI3L1, FCGBP; fragments of any of the foregoing biomarkers that contain an epitope or peptide sequence specific for the biomarker; and combinations thereof;
an analysis tool comprising a statistical analysis engine adapted to generate a representation of a correlation between likelihood of benefit from the therapy and measurements of the biomarkers and clinical parameters, wherein the representation of the correlation is adapted to be executed to generate a result; and
an index computation tool adapted to analyze the result to determine the human subject's likelihood of benefitting from the therapy and represent the result as a numerical probability or a grade or score.

39. A medical diagnostic test system for evaluating likelihood of benefit of a therapy for hepatitis C in a human subject infected with hepatitis C virus (HCV), the system comprising:
a data collection tool adapted to collect biomarker and clinical measurement data representative of measurements of biomarkers and clinical parameters from a human subject, wherein said biomarkers comprise at least one marker selected from the group consisting of:
wherein the at least one biomarker is selected from the group consisting of carnosine dipeptidase 1 (CNDP1); talin 1 (TLN1); pregnancy-zone protein (PZP); apolipoprotein C-IV (APOC4); C-type lectin domain family 3; member B (CLEC3B); apolipoprotein B (APOB); alpha-2-macroglobulin (A2M); lectin, galactoside-binding, soluble, 3 binding protein (LGALS3BP); Fc fragment of IgG binding protein (FCGBP); CD5 molecule-like (CD5L); fragments of any of the foregoing biomarkers that contain an epitope or peptide sequence specific for the biomarker; and combinations thereof;
an analysis tool comprising a statistical analysis engine adapted to generate a representation of a correlation between likelihood of benefit from the therapy and measurements of the biomarkers and clinical parameters, wherein the representation of the correlation is adapted to be executed to generate a result; and
an index computation tool adapted to analyze the result to determine the human subject's likelihood of benefitting from the therapy and represent the result as a numerical probability or a grade or score.

40.-44. (canceled)

45. A method of therapy for HCV infection, the method comprising:
evaluating the likelihood that a human subject infected with HCV will benefit from an HCV therapeutic regimen according to the method of claim 1; and
treating the subject according to the therapeutic regimen if the likelihood of sustained viral response (SVR) six months after cessation of the therapy for the subject of at least 65% probability.

46.-47. (canceled)


SEQUENCE LISTING (42 sequences)

SEQ ID NO: 1 - protein, 16 aa, Homo sapiens
QVEGMEDWKQDSQLQK

SEQ ID NO: 2 - protein, 10 aa, Homo sapiens
VFSLQWGEVK

SEQ ID NO: 3 - protein, 8 aa, Homo sapiens
FPLTNAIK

SEQ ID NO: 4 - protein, 11 aa, Homo sapiens
ALEQDLPVNIK

SEQ ID NO: 5 - protein, 8 aa, Homo sapiens
FLEMAQLH

SEQ ID NO: 6 - protein, 17 aa, Homo sapiens
GDGWLTDPYVLTEVDGK

SEQ ID NO: 7 - protein, 11 aa, Homo sapiens
MMAVAADTLQR

SEQ ID NO: 8 - protein, 20 aa, Homo sapiens
SVVLIPLGAVDDGEHSQNEK

SEQ ID NO: 9 - protein, 20 aa, Homo sapiens
SVVLIPLGAVDDGEHSQNEK

SEQ ID NO: 10 - protein, 11 aa, Homo sapiens
TVFGTEPDMIR

SEQ ID NO: 11 - protein, 17 aa, Homo sapiens
VFQYIDLHQDEFVQTLK

SEQ ID NO: 12 - protein, 17 aa, Homo sapiens
VFQYIDLHQDEFVQTLK

SEQ ID NO: 13 - protein, 8 aa, Homo sapiens
WNYIEGTK

SEQ ID NO: 14 - protein, 19 aa, Homo sapiens
YPSLSIHGIEGAFDEPGTK

SEQ ID NO: 15 - protein, 25 aa, Homo sapiens
AVPPNNSNAAEDDLPTVELQGVVPR

SEQ ID NO: 16 - protein, 14 aa, Homo sapiens
CSVQNGLLGCYPDR

SEQ ID NO: 17 - protein, 8 aa, Homo sapiens
VTFFQNGK

SEQ ID NO: 18 - protein, 12 aa, Homo sapiens
ASHEEVEGLVEK

SEQ ID NO: 19 - protein, 14 aa, Homo sapiens
ELSEALGQIFDSQR

SEQ ID NO: 20 - protein, 14 aa, Homo sapiens
ELSEALGQIFDSQR

SEQ ID NO: 21 - protein, 14 aa, Homo sapiens
ELSEALGQIFDSQR

SEQ ID NO: 22 - protein, 9 aa, Homo sapiens
IDITLSSVK

SEQ ID NO: 23 - protein, 18 aa, Homo sapiens
KTLQALEFHTVPFQLLAR

SEQ ID NO: 24 - protein, 8 aa, Homo sapiens
LASAYGAR

SEQ ID NO: 25 - protein, 8 aa, Homo sapiens
SQLVYQSR

SEQ ID NO: 26 - protein, 9 aa, Homo sapiens
STHTLDLSR

SEQ ID NO: 27 - protein, 16 aa, Homo sapiens
STSSFPCPAGHFNGFR

SEQ ID NO: 28 - protein, 16 aa, Homo sapiens
STSSFPCPAGHFNGFR

SEQ ID NO: 29 - protein, 16 aa, Homo sapiens
STSSFPCPAGHFNGFR

SEQ ID NO: 30 - protein, 12 aa, Homo sapiens
TLQALEFHTVPF

SEQ ID NO: 31 - protein, 17 aa, Homo sapiens
TLQALEFHTVPFQLLAR

SEQ ID NO: 32 - protein, 11 aa, Homo sapiens
VADVTDFEGWK

SEQ ID NO: 33 - protein, 6 aa, Homo sapiens
VEIFYR

SEQ ID NO: 34 - protein, 20 aa, Homo sapiens
YYPYQSFQTPQHPSFLFQDK

SEQ ID NO: 35 - protein, 20 aa, Homo sapiens
YYPYQSFQTPQHPSFLFQDK

SEQ ID NO: 36 - protein, 21 aa, Homo sapiens
YYPYQSFQTPQHPSFLFQDKR

SEQ ID NO: 37 - protein, 21 aa, Homo sapiens
YYPYQSFQTPQHPSFLFQDKR

SEQ ID NO: 38 - protein, 11 aa, Homo sapiens
AVGYLITGYQR

SEQ ID NO: 39 - protein, 15 aa, Homo sapiens
MDPNAAYVNMSNHHR

SEQ ID NO: 40 - protein, 14 aa, Homo sapiens
EGQMESVEAAMSSK

SEQ ID NO: 41 - protein, 14 aa, Homo sapiens
EGQMESVEAAMSSK

SEQ ID NO: 42 - protein, 30 aa, Homo sapiens
MGIQHAYSENADFSGLTEDNGLKLSNAAHK